SEQUENCE LISTING (58 sequences)

SEQ ID NO: 1 - protein, 5 aa, synthetic construct
  REGION 1..5: note = VH-CDR1 for mouse, chimeric and humanized 1C5F12E9 antibodies
TYWMH

SEQ ID NO: 2 - protein, 17 aa, synthetic construct
  REGION 1..17: note = VH-CDR2 for mouse, chimeric and humanized 1C5F12E9 antibodies
VIDPSDSDTTYNQKFKG

SEQ ID NO: 3 - protein, 8 aa, synthetic construct
  REGION 1..8: note = VH-CDR3 for mouse, chimeric and humanized 1C5F12E9 antibodies
SLDGYYDY

SEQ ID NO: 4 - protein, 11 aa, synthetic construct
  REGION 1..11: note = VL-CDR1 for mouse, chimeric and humanized 1C5F12E9 antibodies
RPTENIYSYLA

SEQ ID NO: 5 - protein, 7 aa, synthetic construct
  REGION 1..7: note = VL-CDR2 for mouse, chimeric and humanized 1C5F12E9 antibodies
FARTLAE

SEQ ID NO: 6 - protein, 9 aa, synthetic construct
  REGION 1..9: note = VL-CDR3 for mouse, chimeric and humanized 1C5F12E9 antibodies
QHHYGTPWT

SEQ ID NO: 7 - protein, 117 aa, synthetic construct
  REGION 1..117: note = VH for mouse and chimeric 1C5F12E9 antibodies
QVQLQQPGTELVKPGASVKMSCKASGYTFTTYWMHWVKQRPGQGLEWIGVIDPSDSDTTYNQKFKGKATLTVDTSSSTAYMQLSSLTSEDSAVYYCTRSLDGYYDYWGQGTTLTVSS

SEQ ID NO: 8 - protein, 117 aa, synthetic construct
  REGION 1..117: note = VH for hu1C5F12E9-V1, hu1C5F12E9-V7, and hu1C5F12E9-V13
QVQLVQSGAEVKKPGASVKVSCKASGYTFTTYWMHWVRQAPGQGLEWIGVIDPSDSDTTYNQKFKGRATLTVDTSSSTAYMELSSLRSEDTAVYYCTRSLDGYYDYWGQGTLVTVSS

SEQ ID NO: 9 - protein, 117 aa, synthetic construct
  REGION 1..117: note = VH for hu1C5F12E9-V2 to hu1C5F12E9-V6, hu1C5F12E9-V8 to hu1C5F12E9-V12, and hu1C5F12E9-V14 to hu1C5F12E9-V18
  VARIANT 67: note = misc_feature - Xaa can be Arg or Lys
  VARIANT 68: note = misc_feature - Xaa can be Val or Ala
  VARIANT 72: note = misc_feature - Xaa can be Arg or Val
QVQLVQSGAEVKKPGASVKVSCKASGYTFTTYWMHWVRQAPGQGLEWMGVIDPSDSDTTYNQKFKGXXTMTXDTSTSTVYMELSSLRSEDTAVYYCTRSLDGYYDYWGQGTLVTVSS

SEQ ID NO: 10 - protein, 107 aa, synthetic construct
  REGION 1..107: note = VL for mouse and chimeric 1C5F12E9 antibodies
DIQMTQSPASLSASVGETVTITCRPTENIYSYLAWYQQKQGKSPHLLVYFARTLAEGVPSRFSGSGSGTQFSLKINSLQPEDFGIYYCQHHYGTPWTFGGGTKLEIK

SEQ ID NO: 11 - protein, 107 aa, synthetic construct
  REGION 1..107: note = VL for hu1C5F12E9-V1 to hu1C5F12E9-V18
  VARIANT 43: note = misc_feature - Xaa can Ser or Ala
  VARIANT 48: note = misc_feature - Xaa can be Val or Ile
DIQMTQSPSSLSASVGDRVTITCRPTENIYSYLAWYQQKPGKXPKLLXYFARTLAEGVPSRFSGSGSGTDFTLTISSLQPEDFATYYCQHHYGTPWTFGGGTKVEIK

SEQ ID NO: 12 - protein, 330 aa, synthetic construct
  REGION 1..330: note = Human IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, synthetic construct
  REGION 1..327: note = Human IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 107 aa, synthetic construct [sequence omitted]

SEQ ID NO: 15 - protein, 324 aa, synthetic construct
  REGION 1..324: note = Mouse IgG1 heavy chain constant region
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 107 aa, synthetic construct
  REGION 1..107: note = Mouse kappa light chain constant region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 17 - DNA, 351 bp, synthetic construct
  misc_feature 1..351: note = VH for mouse and chimeric 1C5F12E9 antibodies
  source 1..351: mol_type = other DNA
caggtccaactgcagcagcctgggactgagctggtgaagcctggggcttcagtgaagatgtcctgcaaggcttctggctacaccttcaccacctactggatgcactgggtgaagcagaggcctggacaaggccttgagtggatcggagtgattgatccttctgatagtgatactacctacaatcaaaagttcaagggcaaggccacattgactgtagacacatcctccagcacagcctacatgcagctcagcagcctgacatctgaggactctgcggtctattactgtacaaggtcccttgatggttactacgactactggggccaaggcaccactctcacagtctcctca

SEQ ID NO: 18 - DNA, 351 bp, synthetic construct
  misc_feature 1..351: note = VH for mouse and chimeric 1C5F12E9 antibodies
  source 1..351: mol_type = other DNA
caggtgcagctgcagcagcccggcaccgagctggtgaagcctggcgctagcgtgaagatgtcctgtaaggccagcggctacacattcactacatactggatgcactgggtgaagcagagacctggccagggcctggagtggatcggcgtgatcgaccccagcgactccgataccacctacaaccagaagtttaagggcaaggccaccctgacagtggatacaagctcctccacagcctacatgcagctgtccagcctgacctccgaggattccgccgtgtactactgcacaaggtccctggatggctactacgattactggggccagggcacaaccctgacagtcagcagc

SEQ ID NO: 19 - DNA, 351 bp, synthetic construct
  misc_feature 1..351: note = VH for hu1C5F12E9-V2, hu1C5F12E9-V8, and hu1C5F12E9-V14
  source 1..351: mol_type = other DNA
caggtgcagctggtgcagagcggcgccgaggtgaagaagcctggcgcctccgtgaaggtgtcctgcaaggccagcggctacacattcaccacatactggatgcactgggtgagacaggcccccggccagggactggagtggatgggagtgatcgaccccagcgactccgacacaacctacaaccagaagttcaagggcagggtgacaatgaccagagacaccagcacaagcaccgtgtacatggagctgtcctccctgaggagcgaggacaccgccgtgtactactgcaccagatccctggacggctactacgactactggggccagggcaccctggtgacagtgtccagc

SEQ ID NO: 20 - DNA, 321 bp, synthetic construct
  misc_feature 1..321: note = VL for mouse and chimeric 1C5F12E9 antibodies
  source 1..321: mol_type = other DNA
gacatccagatgactcagtctccagcctccctatctgcatctgtgggagaaactgtcaccatcacatgtcgaccaactgagaatatttacagttatttagcatggtatcagcagaaacagggaaaatctcctcacctcctggtctattttgcaagaaccttagcagaaggtgtgccatcaaggttcagtggcagtggatcaggcacacagttttctctgaagatcaacagcctgcagcctgaagattttgggatttattactgtcaacaccattatggtactccgtggacgttcggtggaggcaccaagctggaaatcaaa

SEQ ID NO: 21 - DNA, 321 bp, synthetic construct
  misc_feature 1..321: note = VL for mouse and chimeric 1C5F12E9 antibodies
  source 1..321: mol_type = other DNA
gacatccagatgacccagtcccccgccagcctgtccgcctctgtgggagagaccgtgaccatcacatgcaggcccaccgagaatatctactcctacctggcctggtatcaacagaagcagggcaagagccctcacctgctggtgtacttcgccaggacactggccgagggcgtgccctctaggttcagcggcagcggctccggcacacagttttccctgaagatcaacagcctgcagcctgaggattttggcatctactactgccagcaccactacggcacaccctggacctttggcggcggcaccaagctggagatcaag

SEQ ID NO: 22 - DNA, 321 bp, synthetic construct
  misc_feature 1..321: note = VL for hu1C5F12E9-V7 - hu1C5F12E9-V12
  source 1..321: mol_type = other DNA
gatatccagatgacacagagccccagcagcctgagcgccagcgtgggagacagggtgacaatcacatgcagacccaccgagaatatctacagctacctggcctggtatcaacagaagcctggcaaggcccccaagctgctgatctacttcgccagaaccctggccgagggcgtgccctctaggttcagcggctccggcagcggcaccgacttcacactgaccatctcctccctgcagcccgaggacttcgccacatactactgccagcaccactacggcacaccttggaccttcggcggcggcacaaaggtggagatcaag

SEQ ID NO: 23 - DNA, 993 bp, synthetic construct
  misc_feature 1..993: note = Human IgG1 heavy chain constant region
  source 1..993: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - DNA, 984 bp, synthetic construct
  misc_feature 1..984: note = Human IgG4 heavy chain constant region
  source 1..984: mol_type = other DNA
gccagcacaaagggcccttccgtgtttcccctggccccctgcagcaggagcacctctgagtccaccgccgccctgggctgtctggtgaaggactactttcccgagcccgtgaccgtgagctggaattccggcgccctgacatccggcgtgcacaccttccccgccgtgctgcagtcctccggcctgtacagcctgagctccgtggtgacagtgccttcctcctccctgggcaccaagacctacacatgtaatgtggatcacaagcccagcaacacaaaggtggataagagagtggagtccaagtacggccctccttgccctccctgtcctgccccagagttcctgggcggcccctctgtgttcctgttcccccctaagcccaaggacacactgatgatctccaggacccctgaggtgacctgcgtggtggtggacgtgagccaggaggaccctgaggtgcagttcaattggtacgtggatggcgtggaggtgcacaatgccaagacaaagcccagagaggagcagtttaattccacatacagggtggtgtccgtgctgaccgtgctgcaccaggattggctgaacggcaaggagtacaagtgtaaggtgagcaacaagggcctgccttcctccatcgagaagacaatcagcaaggccaagggccagcctagggagccccaggtgtacacactgcctcccagccaggaggagatgaccaagaaccaggtgagcctgacctgcctggtgaagggcttctaccctagcgacatcgccgtggagtgggagtccaacggccagcccgagaataactacaagacaacaccccccgtgctggattccgatggcagcttctttctgtactccaggctgaccgtggataagagcaggtggcaggagggcaatgtgttcagctgctccgtgatgcacgaggccctgcacaatcactacacccagaagagcctgtccctgagcctgggcaagtga

SEQ ID NO: 25 - DNA, 324 bp, synthetic construct
  misc_feature 1..324: note = Human kappa light chain constant region
  source 1..324: mol_type = other DNA
cgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 26 - DNA, 975 bp, synthetic construct
  misc_feature 1..975: note = Mouse IgG1 heavy chain constant region
  source 1..975: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 27 - DNA, 321 bp, synthetic construct
  misc_feature 1..321: note = Mouse kappa light chain constant region
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgactaaggacgagtatgaacgacataacagctatacctgtgaggccactcacaagacatcaacttcacccattgtcaagagcttcaacaggggagagtgt

SEQ ID NO: 28 - protein, 141 aa, synthetic construct
  REGION 1..141: note = Recombinant human TSLP-his
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQHHHHHHHHHH

SEQ ID NO: 29 - protein, 140 aa, synthetic construct
  REGION 1..140: note = Recombinant cynomolgus monkey TSLP-his
MYDFTNCDFQKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQLEHHHHHH

SEQ ID NO: 30 - protein, 446 aa, synthetic construct
  REGION 1..446: note = human TSLPR-Fc
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, synthetic construct
  REGION 1..448: note = human IL7Ra-Fc
ESGYAQNGDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 363 aa, synthetic construct
  REGION 1..363: note = human TSLP-Fc
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 34 - protein, 459 aa, Homo sapiens [sequence omitted]

SEQ ID NO: 35 - protein, 448 aa, synthetic construct
  REGION 1..448: note = Heavy chain of Tezepelumab
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 214 aa, synthetic construct
  REGION 1..214: note = Light chain of Tezepelumab
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 5 aa, synthetic construct
  REGION 1..5: note = VH-CDR1 for mouse antibody D1D8H9F7
SYYMN

SEQ ID NO: 38 - protein, 17 aa, synthetic construct
  REGION 1..17: note = VH-CDR2 for mouse antibody D1D8H9F7
QIYPGDGETNYNGKFKD

SEQ ID NO: 39 - protein, 12 aa, synthetic construct
  REGION 1..12: note = VH-CDR3 for mouse antibody D1D8H9F7
AGYSDYVYAMDY

SEQ ID NO: 40 - protein, 12 aa, synthetic construct
  REGION 1..12: note = VL-CDR1 for mouse antibody D1D8H9F7
SVSSSITSSNLH

SEQ ID NO: 41 - protein, 7 aa, synthetic construct
  REGION 1..7: note = VL-CDR2 for mouse antibody D1D8H9F7
GTSNLAS

SEQ ID NO: 42 - protein, 8 aa, synthetic construct
  REGION 1..8: note = VL-CDR3 for mouse antibody D1D8H9F7
QQWSSFPL

SEQ ID NO: 43 - protein, 121 aa, synthetic construct
  REGION 1..121: note = VH for mouse antibody D1D8H9F7
QVQLQQSGADLVRPGSSVKISCKTSGFAFSSYYMNWVKQRPGQGLEWIGQIYPGDGETNYNGKFKDKATLTADKSSSTVYMQLSSLTSEDSAVYFCARAGYSDYVYAMDYWGQGTSVTVSS

SEQ ID NO: 44 - protein, 108 aa, synthetic construct
  REGION 1..108: note = VL for mouse antibody D1D8H9F7
EIVLTQSPALMAASPGEKVTIICSVSSSITSSNLHWYQQKSETSPKPWIYGTSNLASGVPVRFSGSGSGTSYSLTISSMEAEDAATYYCQQWSSFPLTFGSGTKLEIK

SEQ ID NO: 45 - DNA, 363 bp, synthetic construct
  misc_feature 1..363: note = VH for mouse antibody D1D8H9F7
  source 1..363: mol_type = other DNA
caggttcagctgcagcagtctggggctgacctggtgaggcctgggtcctcagtgaagatttcctgcaagacttctggctttgcattcagtagctactatatgaactgggtgaagcagaggcctggacagggtcttgagtggattggacagatttatcctggagatggtgaaactaactacaatggaaagttcaaggataaagccacactgactgcagacaaatcctccagcacagtctacatgcagctcagcagcctaacatctgaggactctgcggtctacttctgtgcaagagcgggttatagtgactacgtttatgctatggactactggggtcaaggaacctcagtcaccgtctcctca

SEQ ID NO: 46 - DNA, 324 bp, synthetic construct
  misc_feature 1..324: note = VL for mouse antibody D1D8H9F7
  source 1..324: mol_type = other DNA
gaaattgtgctcacccagtctccagcactcatggctgcatctccaggggagaaggtcaccatcatctgcagtgtcagctcaagtataacttccagcaacctgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagtttcccactcacgttcggctcggggacaaagttggaaataaaa

SEQ ID NO: 47 - protein, 7 aa, synthetic construct
  REGION 1..7: note = VH-CDR1 for mouse antibody D1C2H1H1
TYGMGIG

SEQ ID NO: 48 - protein, 16 aa, synthetic construct
  REGION 1..16: note = VH-CDR2 for mouse antibody D1C2H1H1
SIWWDDNRYFNAALKS

SEQ ID NO: 49 - protein, 11 aa, synthetic construct
  REGION 1..11: note = VH-CDR3 for mouse antibody D1C2H1H1
IEDGYYSYHSY

SEQ ID NO: 50 - protein, 15 aa, synthetic construct
  REGION 1..15: note = VL-CDR1 for mouse antibody D1C2H1H1
KASQSVDFDGDSYMN

SEQ ID NO: 51 - protein, 7 aa, synthetic construct
  REGION 1..7: note = VL-CDR2 for mouse antibody D1C2H1H1
AASNLVS

SEQ ID NO: 52 - protein, 9 aa, synthetic construct
  REGION 1..9: note = VL-CDR3 for mouse antibody D1C2H1H1
QQIDEDPYT

SEQ ID NO: 53 - protein, 121 aa, synthetic construct
  REGION 1..121: note = VH for mouse antibody D1C2H1H1
QVTLKESGPGILQPSQTLSLTCSFSGFSLSTYGMGIGWIRLPSGKGLEWLASIWWDDNRYFNAALKSRLTISKDASKNQVFLKIASVDTADTATYYCARIEDGYYSYHSYWGQGTLVTVSA

SEQ ID NO: 54 - protein, 111 aa, synthetic construct
  REGION 1..111: note = VL for mouse antibody D1C2H1H1
DIVLTQSPASLAVSLGQRATISCKASQSVDFDGDSYMNWFQQKPGQPPKLLIYAASNLVSGIPARFSGSGSGTDFTLNIHPVEEEDAATYYCQQIDEDPYTFGGGTKLEIE

SEQ ID NO: 55 - DNA, 363 bp, synthetic construct
  misc_feature 1..363: note = VH for mouse antibody D1C2H1H1
  source 1..363: mol_type = other DNA
caggttactctgaaagagtctggccctgggatattgcagccctcccagaccctcagtctgacttgttctttctctgggttttcactgagcacttatggcatgggtataggctggattcgtctgccttcggggaagggtctggagtggctggcaagcatttggtgggatgataatagatactttaacgctgccctgaagagccggctcactatctccaaggatgcctccaaaaaccaggtattcctcaagatcgccagtgtggacactgcagatactgccacatattattgtgctcgaatagaggacggctattatagttatcactcttactggggccaagggactctggtcactgtctctgca

SEQ ID NO: 56 - DNA, 333 bp, synthetic construct
  misc_feature 1..333: note = VL for mouse antibody D1C2H1H1
  source 1..333: mol_type = other DNA
gacattgtgctgacccaatctccagcttctttggctgtgtctctagggcagagggccaccatctcctgcaaggccagccaaagtgttgattttgatggtgatagttatatgaactggttccaacagaaaccaggacagccacccaaactcctcatctatgctgcatccaatctagtatctgggatcccagccaggtttagtggcagtgggtctgggacagacttcaccctcaacatccatcctgtggaggaggaggatgctgcaacctattactgtcagcaaattgatgaggatccgtacacgttcggaggggggaccaagctggaaatagaa

SEQ ID NO: 57 - protein, 330 aa, synthetic construct [sequence omitted]

SEQ ID NO: 58 - DNA, 993 bp, synthetic construct
  misc_feature 1..993: note = Human IgG1 heavy chain constant region
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2026/0167711 A1
(43) Pub. Date: Jun. 18, 2026

(54) ANTIBODIES BINDING TSLP AND USES THEREOF

(71) Applicant: BIOSION INC, Nanjing (CN)

(72) Inventors: Mingjiu CHEN, Hockessin, DE (US); Wei TAN, Nanjing (CN); Cathy Xiaoyan ZHONG, Nanjing (CN); Zhengping ZHANG, Lianyungang (CN); Xiaofang ZOU, Lianyungang (CN); Wei SONG, Lianyungang (CN); Hongjiang XU, Lianyungang (CN)

(21) Appl. No.: 19/388,619

(22) Filed: Nov. 13, 2025

Related U.S. Application Data
(63) Continuation of application No. 17/636,089, now Pat. No. 12,479,912.
(60) Provisional application No. 62/895,984, filed on Sep. 4, 2019.

(51) Int. Cl. C07K 16/24

(52) U.S. Cl. CPC C07K 16/244; C07K 2317/24; C07K 2317/92

(57) ABSTRACT

Disclosed is an isolated monoclonal antibody that specifically binds human TSLP, or the antigen-binding portion thereof. A nucleic acid molecule encoding the antibody, an expression vector, a host cell and a method for expressing the antibody are also provided. The present disclosure further provides a bispecific molecule, an immunoconjugate, a CAR-immune cell, an oncolytic virus and a pharmaceutical composition comprising the antibody, as well as a treatment method using an anti-TSLP antibody of the disclosure.

### RELATED APPLICATIONS AND INCORPORATION BY REFERENCE

This application is a continuation of U.S. patent application Ser. No. 17/636,089, filed Feb. 17, 2022, which is a U.S. National Stage filing under 35 U.S.C. § 371 of International PCT Application No. PCT/CN2020/113289, filed Sep. 3, 2020, which claims the benefit of priority to U.S. Provisional Application No. 62/895,984 filed Sep. 4, 2019, each of which is incorporated herein by reference in its entirety.

### INCORPORATION BY REFERENCE OF SEQUENCE LISTING

The instant application contains a Sequence Listing which has been submitted electronically in XML file format and is hereby incorporated by reference in its entirety. Said XML copy, created on Dec. 23, 2025, is named 145688-005911_SL.xml and is 88,922 bytes in size.

The foregoing application, and all documents cited therein or during its prosecution (“appIn cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### FIELD OF THE INVENTION

The present disclosure relates generally to an isolated monoclonal antibody, particularly a mouse, chimeric or humanized monoclonal antibody, or the antigen-binding portion thereof, that specifically binds to human TSLP with high affinity and functionality. A nucleic acid molecule encoding the antibody or antigen-binding portion thereof, an expression vector, a host cell and a method for expressing the antibody or antigen-binding portion thereof are also provided. The present disclosure further provides an immunoconjugate, a bispecific molecule, a chimeric antigen receptor, an oncolytic virus, and a pharmaceutical composition comprising the antibody or the antigen-binding portion thereof, as well as a diagnostic and treatment method using an anti-TSLP antibody or antigen-binding portion thereof of the disclosure.

### BACKGROUND OF THE INVENTION

Thymic stromal lymphopoietin (TSLP) is an epithelially derived cytokine. It is closely related to IL-7 and binds to TSLPR, a heterodimer of the IL-7 receptor alpha chain and the TSLP receptor chain. TSLP mRNA is expressed predominantly by epithelial cells in the thymus, lung, skin, intestine and tonsils, as well as stromal cells and mast cells, while TSLPR mRNA is found on many immune cell types, including dendritic cells (DCs), T cells, B cells, mast cells, NK cells and monocytes, as well as in tissues like heart, skeletal muscle, kidney and liver (Rui He et al., (2010) Annals of the New Youk Academy ofSciences 1183:13-24; Quentmeier H et al., (2001) Leukemia 15(8): 1286-1292; Rimoldi M et al., (2005) Nature immunology 6(5): 507-514).

The signaling pathways triggered by TSLP have been extensively studied. TSLP induces DC polarization to drive T helper (Th)2 cell differentiation and Th2 cytokine production during the induction phase of the immune response, and also directly promotes T cell expansion and amplifies Th2 cytokine secretion. Accordingly, TSLP is believed to be a master regulator of Th2 driven inflammation, and upregulation of TSLP is linked to the pathogenesis of Th2-related diseases, such as atopic dermatitis, and asthma (Rui He et al., (2010) supra; Ito T et al., (2005) The Journal of Experimental Medicine 202(9): 1213-1223; He R et al., (2008) Proc Natl Acad Sci USA 105(33):11875-11880). On the other hand, TSLP mediates several immune homeostatic functions in the gut and the thymus. For example, TSLP is upregulated in gut epithelial cell lines upon bacterial stimulation in a strain-dependent fashion, which synergizes with transforming growth factor-beta to promote Treg cell differentiation. TSLP is also produced by primary human intestinal epithelial cells for the conditioning of CD103+ DCs to a tolerogenic phenotype (Katerina Tsilingiri et al., (2017) Cellular and Molecular Gastroenterology and Hepatology 3(2): 174-182; Zeuthen L H et al., (2008) Immunology 123:197-208; Iliev I D et al., (2009) Gut 58:1481-1489).

The dual role of TSLP on the immune system leads to the discovery of two isoforms, a long isoform and a short isoform composed of the last 63 amino acid residues of the longer one. These two are controlled by different promoter regions, and are expressed depending on the context, tissue and stimulus (Harada M et al., (2011) Amrican Journal of Respiratory Cell and Molecular Biology 44:787-793). Long isoform expression was upregulated while the short isoform expression was downregulated in human intestinal epithelial cells in response to highly immunogenic microbial strains, whereas the opposite expression pattern was observed after challenge with a commensal E. coli strain. Further, TSLP expression on healthy barrier surfaces is limited to the short isoform, and long TSLP is only upregulated in oral mucosal lesions after tobacco exposure (Katerina Tsilingiri et al., (2017) supra; The isoform expression pattern has also been investigated in some TSLP-related diseases. For example, over-expression of long TSLP was observed in asthma, ulcerative colitis, atopic dermatitis and psoriasis, and reduced expression of long TSLP was found in celiac disease. And the expression of short TSLP was downregulated in Crohn's disease, celiac disease and atopic dermatitis (Katerina Tsilingiri et al., (2017) supra′, Fomasa G et al., (2015) J Allergy Clin Immunol 136:413-422).

TSLP emerges as a clinical target because of its relevance to diseases mentioned above. The restoring of short TSLP expression was proposed to treat refractory celiac disease (Biancheri P et al., (2015) Gut 65:1670-1680). A human monoclonal antibody Tezepelumab was designed for the treatment of asthma and atopic dermatitis and has been proved to reduce the annual asthma exacerbation rate in Phase II trial compared to placebo (Jonathan Corren et al., (2017) The New England Journal of Medicine 377(10): 936-946).

More antibodies with better pharmaceutical properties are increasingly desired and pursued, including those specific to only the long TSLP with minimal interaction with the short isoform, which may have and result in improved clinical outcome.

Citation or identification of any document in this application is not an admission that such document is available as prior art to the present disclosure.

### SUMMARY OF THE INVENTION

The present disclosure provides an isolated monoclonal antibody, for example, a mouse, human, chimeric or humanized monoclonal antibody, or an antigen-binding portion thereof, that binds to TSLP (e.g., the human TSLP, and monkey TSLP) and has comparable, if not higher, binding affinity to TSLP and blocking activity on TSLP-TSLPR/IL7R interaction as compared to prior art anti-TSLP antibodies such as Tezepelumab.

The antibody or antigen-binding portion thereof of the disclosure can be used for a variety of applications, including detection of the TSLP protein, and treatment and prevention of TSLP associated diseases, such as asthma, ulcerative colitis, atopic dermatitis and psoriasis.

Accordingly, in one aspect, the disclosure pertains to an isolated monoclonal antibody (e.g., a mouse, chimeric or humanized antibody), or an antigen-binding portion thereof, that binds TSLP, having a heavy chain variable region that may comprise a VH-CDR1 region, a VH-CDR2 region and a VH-CDR3 region, wherein the VH-CDR1 region, the VH-CDR2 region and the CDR3 region may comprise amino acid sequences set forth in (1) SEQ ID NOs: 1, 2 and 3, respectively; (2) SEQ ID Nos: 37, 38, and 39, respectively; or (3) SEQ ID Nos: 47, 48, and 49, respectively.

The isolated monoclonal antibody, or the antigen-binding portion thereof, of the present disclosure may comprise a heavy chain variable region comprising an amino acid sequence set forth in SEQ ID NOs: 7, 8, 9 (X1=R, X2=V, X3=R; X1=R, X2=V, X3=V; X1=R, X2=A, X3=R; X1=K, X2=A, X3=R; or X1=K, X2=A, X3=V), 43 or 53, wherein the antibody or antigen-binding portion thereof binds to TSLP. The amino acid sequence of SEQ ID NO.:7 may be encoded by the nucleotide sequence of SEQ ID NOs: 17 or 18, and the amino acid sequences set forth in SEQ ID NO.: 9 (X1=R, X2=V, X3=R), 43 and 53 may be encoded by the nucleotide sequences of SEQ ID NOs: 19, 45 and 55, respectively.

The isolated monoclonal antibody, or the antigen-binding portion thereof, of the present disclosure, that binds TSLP, may comprise a light chain variable region that comprises a VL-CDR1 region, a VL-CDR2 region and a VL-CDR3 region, wherein the VL-CDR1 region, the VL-CDR2 region, and the VL-CDR3 region may comprise amino acid sequences set forth in (1) SEQ ID NOs: 4, 5 and 6, respectively; (2) SEQ ID NOs: 40, 41 and 42, respectively; or (3) SEQ ID NOs: 50, 51 and 52, respectively.

The isolated monoclonal antibody, or the antigen-binding portion thereof, of the present disclosure may comprise a light chain variable region comprising an amino acid sequence set forth in SEQ ID NOs: 10, 11 (X1=S, X2=V; X1=A, X2=I; or X1=S, X2=I), 44 or 54, wherein the antibody or antigen-binding portion thereof binds to TSLP. The amino acid sequence of SEQ ID NO.: 10 may be encoded by the nucleotide sequences of SEQ ID NOs: 20 or 21, and the amino acid sequences set forth in SEQ ID NO.: 11 (X1=A, X2=I), 44 and 54 may be encoded by nucleotide sequences of SEQ ID NOs: 22, 46 and 56, respectively.

In the isolated monoclonal antibody, or the antigen-binding portion thereof, of the present disclosure, the VH-CDR1, VH-CDR2, VH-CDR3, VL-CDR1, VL-CDR2 and VL-CDR3 may comprise amino acid sequences set forth in (1) SEQ ID NOs: 1, 2, 3, 4, 5 and 6, respectively; (2) SEQ ID NOs: 37, 38, 39, 40, 41 and 42, respectively; or (3) SEQ ID NOs: 47, 48, 49, 50, 51 and 52, respectively, wherein the antibody or antigen-binding portion thereof binds to TSLP.

The isolated monoclonal antibody, or the antigen-binding portion thereof, of the present disclosure may comprises a heavy chain variable region and a light chain variable region that may comprise amino acid sequences set forth in (1) SEQ ID NOs: 7 and 10, respectively; (2) SEQ ID NOs: 8 and 11 (X1=S, X2=V), respectively; (3) SEQ ID NOs: 9 (X1=R, X2=V, X3=R) and 11 (X1=S, X2=V), respectively; (4) SEQ ID NOs: 9 (X1=R, X2=V, X3=V) and 11 (X1=S, X2=V), respectively; (5) SEQ ID NOs: 9 (X1=R, X2=A, X3=R) and 11 (X1=S, X2=V), respectively; (6) SEQ ID NOs: 9 (X1=K, X2=A, X3=R) and 11 (X1=S, X2=V), respectively; (7) SEQ ID NOs: 9 (X1=K, X2=A, X3=V) and 11 (X1=S, X2=V), respectively; (8) SEQ ID NOs: 8 and 11 (X1=A, X2=I), respectively; (9) SEQ ID NOs: 9 (X1=R, X2=V, X3=R) and 11 (X1=A, X2=I), respectively; (10) SEQ ID NOs: 9 (X1=R, X2=V, X3=V) and 11 (X1=A, X2=I), respectively; (11) SEQ ID NOs: 9 (X1=R, X2=A, X3=R) and 11 (X1=A, X2=I), respectively; (12) SEQ ID NOs: 9 (XI=K, X2=A, X3=R) and 11 (X1=A, X2=I), respectively; (13) SEQ ID NOs: 9 (X1=K, X2=A, X3=V) and 11 (X1=A, X2=I), respectively; (14) SEQ ID NOs: 8 and 11 (X1=S, X2=I), respectively; (15) SEQ ID NOs: 9 (X1=R, X2=V, X3=R) and 11 (X1=S, X2=I), respectively; (16) SEQ ID NOs: (X1=R, X2=V, X3=V) and 11 (X1=S, X2=I), respectively; (17) SEQ ID NOs: 9 (X1=R, X2=A, X3=R) and 11 (X1=S, X2=I), respectively; (18) SEQ ID NOs: 9 (X1=K, X2=A, X3=R) and 11 (X1=S, X2=I), respectively; (19) SEQ ID NOs: 9 (X1=K, X2=A, X3=V) and 11 (X1=S, X2=I), respectively; (20) SEQ ID NOs: 43 and 44, respectively; or (21) SEQ ID NOs: 53 and 54, respectively, wherein the antibody or antigen-binding portion thereof binds to TSLP.

The isolated monoclonal antibody, or the antigen-binding portion thereof, of the present disclosure may comprise a heavy chain and a light chain, wherein the heavy chain may comprise a heavy chain variable region and a heavy chain constant region, the light chain may comprise a light chain variable region and a light chain constant region. The heavy chain constant region may comprise human IgGl constant region having the amino acid sequences set forth in SEQ ID NOs: 12 or 57, or human IgG4 constant region having the amino acid sequence set forth in SEQ ID NO: 13, or a fragment thereof. The light chain constant region may comprise human kappa constant region having the amino acid sequence set forth in SEQ ID NO: 14, or a fragment thereof. The heavy chain constant region may also be mouse IgGl constant region having the amino acid sequence of SEQ ID NO.: 15, and the light chain constant region may be mouse kappa constant region having the amino acid sequence of SEQ ID NO.: 16. The amino acid sequences of SEQ ID NOs: 12 to 16, and 57 may be encoded by the nucleotide sequences of SEQ ID NOs: 23 to 27, and 58, respectively.

The antibody of the present disclosure may comprise or consist of two heavy chains and two light chains connected by disulfide bonds, wherein each heavy chain comprises the heavy chain constant region, heavy chain variable region or CDR sequences mentioned above, and each light chain comprises the light chain constant region, light chain variable region or CDR sequences mentioned above, wherein the C-terminus of the heavy chain variable region is linked to the N-terminus of the heavy chain constant region, and the C-terminus of the light chain variable region is linked to the N-terminus of the light chain constant region. The antibody of the disclosure can be a full-length antibody, for example, of an IgG1, IgG2 or IgG4 isotype. The antibody of the present disclosure in other embodiments may be a single chain variable fragment (scFv) antibody, or antibody fragments, such as Fab or F(ab′)2 fragments.

The antibody, or the antigen-binding portion thereof, of the present disclosure has comparable or higher binding affinity to TSLP and blocking activity on TSLP-TSLPR/IL7R interaction as compared to prior art anti-TSLP antibodies such as Tezepelumab.

The disclosure also provides a bispecific molecule comprising an antibody, or an antigen-binding portion thereof, of the disclosure, linked to a second functional moiety (e.g., a second antibody) having a different binding specificity than said antibody, or antigen-binding portion thereof. The bispecific molecule may bind to TSLP and another disease specific protein such as IgE. The disclosure also provides an immunoconjugate comprising an antibody or an antigen-binding portion thereof of the invention, linked to a therapeutic agent, such as a cytotoxin, cytotoxic drug, etc. In another aspect, the antibody or an antigen binding portions thereof of the present invention can be made into part of a chimeric antigen receptor (CAR), and the present disclosure also relates to an immune cell, e.g., a T cell or a NK cell, comprising the CAR. The antibody or an antigen binding portions thereof of the present invention can also be encoded by or used in conjunction with an oncolytic virus.

Compositions comprising an antibody, or an antigen-binding portion thereof, or an immunoconjugate, a bispecific molecule, a CAR-immune cell, or an oncolytic virus of the invention, and a pharmaceutically acceptable carrier, are also provided.

Nucleic acid molecules encoding the antibodies, or antigen-binding portions thereof, of the disclosure are also encompassed by the disclosure, as well as expression vectors comprising such nucleic acids and host cells comprising such expression vectors or having its genome integrated with the polynucleotide encoding the antibody or the antigen-binding portion thereof. A method for preparing an anti-TSLP antibody or an antigen-binding portion thereof using the host cell comprising the expression vector is also provided, comprising steps of (i) expressing the antibody or antigen-binding portion thereof in the host cell and (ii) isolating the antibody or antigen-binding portion thereof from the host cell or its cell culture.

In yet another aspect, the disclosure provides a method of treating asthma, ulcerative colitis, atopic dermatitis or psoriasis in a subject, comprising administering to the subject a therapeutically effective amount of the antibody, or antigen-binding portion thereof, of the disclosure. In some embodiments, the method comprises administering a composition, a bispecific molecule, an immunoconjugate, a CAR-immune cell, or an antibody-encoding or antibody-bearing oncolytic virus of the disclosure, or alternatively a nucleic acid molecule capable of expressing the same in the subject. The bispecific molecule may bind to TSLP and another disease specific protein, such as IgE, IL4, IL13 or IL-5 in asthma treatment. In some embodiments, at least one additional antibody can be administered with the antibody, or an antigen-binding portion thereof, of the disclosure, such as an anti-IgE antibody, an anti-IL4 antibody, an anti-IL4R antibody, an anti-IL13 antibody, an anti-IL13R antibody, an anti-IL-5 antibody, an anti-IL5R antibody, and/or an anti-TSLPR antibody. In some embodiments, at least one additional drug can be administered with the antibody, or an antigen-binding portion thereof, of the disclosure, such as an anti-asthma drug, an anti-ulcerative colitis drug, an anti-atopic dermatitis drug, or an anti-psoriasis drug.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1B show the binding capacities of mouse antibodies 1C5F12E9 (A), D1C2H1H1 and D1D8H9F7 (B) to human TSLP in a capture ELISA.

FIGS. 2A-2B show the binding capacities of mouse antibodies 1C5F12E9 (A), D1C2H1H1 and D1D8H9F7 (B) to cynomolgus TSLP in an indirect ELISA.

FIGS. 3A-3B show the blocking abilities of mouse antibodies 1C5F12E9 (A), D1C2H1H1 and D1D8H9F7 (B) on binding of human TSLP to TSLPR/IL7R in a competitive ELISA.

FIGS. 4A-4B show the abilities of mouse antibodies 1C5F12E9 (A), D1C2H1H1 and D1D8H9F7 (B) to block Benchmark Tezepelumab binding to human TSLP in a competitive ELISA.

FIGS. 5A-5B show the blocking abilities of mouse antibodies 1C5F12E9 (A), D1C2H1H1 and D1D8H9F7 (B) on binding of human TSLP to engineered BAF3 cells expressing human TSLPR and IL7R in a cell-based ligand blocking FACS assay.

FIGS. 6A-6B show the inhibitory effects of 1C5F12E9 (A), D1C2H1H1 and D1D8H9F7 (B) on BAF3 cell survival and proliferation in a cell-based functional assay.

FIG. 7 shows the binding capacity of chimeric 1C5F12E9 antibody to human TSLP in a capture ELISA.

FIG. 8 shows the blocking ability of chimeric 1C5F12E9 antibody on binding of human TSLP to engineered BAF3 cells expressing human TSLPR and IL7R in a cell-based blocking FACS assay.

FIG. 9 shows the inhibitory effect of chimeric 1C5F12E9 antibody on BAF3 cell survival and proliferation in a cell-based functional assay.

FIG. 10 shows the binding capacities of humanized antibodies hulC5F12E9-V8 and hulC5F12E9-V14 to human TSLP in a capture ELISA.

FIG. 11 shows the binding capacities of humanized antibodies hulC5F12E9-V8 and hulC5F12E9-V14 to cynomolgus TSLP in an indirect ELISA.

FIG. 12 shows the blocking abilities of humanized antibodies hulC5F12E9-V8 and hulC5F12E9-V14 on binding of human TSLP to TSLPR/IL7R in a competitive ELISA.

FIG. 13 shows the abilities of humanized antibodies hulC5F12E9-V8 and hulC5F12E9-V14 to block Benchmark Tezepelumab binding to human TSLP in a competitive ELISA.

FIG. 14 shows the blocking abilities of humanized antibodies hulC5F12E9-V8 and hulC5F12E9-V14 on binding of human TSLP to engineered BAF3 cells expressing human TSLPR and IL7R in a cell-based ligand blocking FACS assay.

FIGS. 15A and 15B show the inhibitory effects of humanized antibodies hulC5F12E9-V8 (A) and hulC5F12E9-V14 (B) on BAF3 cell survival and proliferation in a cell-based functional assay.

FIG. 16 shows the binding capacities of humanized antibodies hulC5F12E9-V8 (IgGl), hulC5F12E9-V8 (IgG4), hulC5F12E9-V14 (IgGl) and hulC5F12E9-V14 (IgG4) to human TSLP in a capture ELISA test.

FIG. 17 shows the blocking abilities of humanized antibodies hulC5F12E9-V8 (IgGl), hulC5F12E9-V8 (IgG4), hulC5F12E9-V14 (IgGl) and hulC5F12E9-V14 (IgG4) on human TSLP-TSLPR/IL7R binding in a competitive ELISA.

FIG. 18 shows the abilities of humanized antibodies hulC5F12E9-V8 (IgGl), hulC5F12E9-V8 (IgG4), hulC5F12E9-V14 (IgGl) and hulC5F12E9-V14 (IgG4) to block Benchmark-human TSLP binding in a competitive ELISA.

FIG. 19 shows the blocking abilities of humanized antibodies hulC5F12E9-V8 (IgGl), hulC5F12E9-V8 (IgG4), hulC5F12E9-V14 (IgGl) and hulC5F12E9-V14 (IgG4) on binding of human TSLP to engineered BAF3 cells expressing human TSLPR and IL7R in a cell-based ligand blocking FACS assay.

FIGS. 20A and 20B show the inhibitory effects of humanized antibodies hulC5F12E9-V8 (IgGl) and hulC5F12E9-V8 (IgG4) (A), and hulC5F12E9-V14 (IgGl) and hulC5F12E9-V14 (IgG4) (B) on BAF3 cell survival and proliferation in a cell-based functional assay.

FIGS. 21A and 21B show the blocking abilities of humanized antibodies hulC5F12E9-V8 (IgGl) and hulC5F12E9-V8 (IgG4) (A), and hulC5F12E9-V14 (IgGl) and hulC5F12E9-V14 (IgG4) (B) on interaction of human TSLP with engineered HEK293T cells in a cell-based reporter assay.

FIG. 22 shows the protein thermal shift assay results of hulC5F12E9-V8 (IgGl), hulC5F12E9-V8 (IgG4), hulC5F12E9-V14 (IgGl), hulC5F12E9-V14 (IgG4) antibodies.

### DETAILED DESCRIPTION OF THE INVENTION

To ensure that the present disclosure may be more readily understood, certain terms are first defined. Additional definitions are set forth throughout the detailed description.

The term “TSLP” refers to thymic stromal lymphopoietin. The term “TSLP” comprises variants, isoforms, homologs, orthologs and paralogs. For example, an antibody specific for a human TSLP protein may, in certain cases, cross-react with a TSLP protein from a species other than human, such as monkey. In other embodiments, an antibody specific for a human TSLP protein may be completely specific for the human TSLP protein and exhibit no cross-reactivity to other species or of other types, or may cross-react with TSLP from certain other species but not all other species.

The term “human TSLP” refers to a TSLP protein having an amino acid sequence from a human, such as the amino acid sequence of human TSLP having a Genbank accession number of NP_149024.1. The terms “monkey or rhesus TSLP” and “mouse TSLP” refer to monkey and mouse TSLP sequences, respectively, e.g. those with the amino acid sequences having Genbank Accession Nos. NP_001100503.1 and NP_067342.1, respectively.

The term “antibody” as referred to herein includes whole antibodies and any antigen binding fragment (i.e., “antigen-binding portion”) or single chains thereof. Whole antibodies are glycoproteins comprising two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., a TSLP protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989)Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, VL and V_{H}, are coded by separate genes, they can be joined, using recombinant methods, by a synthetic linker that enables them to be made as a single protein chain in which the V_{H} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a TSLP protein is substantially free of antibodies that specifically bind antigens other than TSLP proteins). An isolated antibody that specifically binds a human TSLP protein may, however, have cross-reactivity to other antigens, such as TSLP proteins from other species. Moreover, an isolated antibody can be substantially free of other cellular material and/or chemicals. The terms “monoclonal antibody” or “monoclonal antibody composition” as used herein refer to a preparation of antibody molecules of single molecular composition. A monoclonal antibody composition displays a single binding specificity and affinity for a particular epitope.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “isotype” refers to the antibody class (e.g., IgM or IgGl) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human TSLP” is intended to refer to an antibody that binds to human TSLP protein (and possibly a TSLP protein from one or more non-human species) but does not substantially bind to non-TSLP proteins. Preferably, the antibody binds to human TSLP protein with “high affinity”, namely with a K_{D} of 5.0×10⁻⁸ M or less, more preferably 10×10⁻⁸ M or less, and more preferably 7.0×10⁻⁹ M or less.

The term “does not substantially bind” to a protein or cells, as used herein, means does not bind or does not bind with a high affinity to the protein or cells, i.e. binds to the protein or cells with a K_{D} of 10×10⁻⁶ M or more, more preferably 1.0×10⁻⁵ M or more, more preferably 10×10⁻⁴ M or more, more preferably 1.0×10⁻³ M or more, even more preferably 10×10⁻² M or more.

The term “high affinity” for an IgG antibody refers to an antibody having a K_{D} of 10×10⁻⁶ M or less, more preferably 5.0×10⁻⁸ M or less, even more preferably 1.0×10⁻⁸ M or less, even more preferably 7.0×10⁻⁹ M or less and even more preferably 1.0×10⁻⁹ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 108 M or less.

The term -K_{assoc} or Kₐ, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “subject” includes any human or nonhuman animal. The term “nonhuman animal” includes all vertebrates, e.g., mammals and non-mammals, such as nonhuman primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody of the present disclosure sufficient to prevent or ameliorate the symptoms associated with a disease or condition (such as a cancer) and/or lessen the severity of the disease or condition. A therapeutically effective amount is understood to be in context to the condition being treated, where the actual effective amount is readily discerned by those of skill in the art.

Various aspects of the disclosure are described in further detail in the following subsections.

Anti-TSLP Antibodies Having Increased Binding Affinity to human TSLP and Better Blocking Capacity on TSLP-TSLPR/IL7R Interaction

The exemplary antibody, or the antigen-binding portion thereof, of the disclosure specifically binds to human or cynomolgus TSLP with comparable, if not better, binding affinity as compared to previously described anti-TSLP antibodies, such as Tezepelumab. The exemplary antibody, or the antigen-binding portion thereof, of the disclosure may also block human TSLP-TSLPR/IL7R interaction, and its blocking activity is better than prior art anti-TSLP antibodies.

Preferred antibodies of the disclosure are humanized monoclonal antibodies. Additionally, or alternatively, the antibodies can be, for example, chimeric monoclonal antibodies.

### Monoclonal Anti-TSLP Antibody

The antibody or the antigen-binding portion thereof of the disclosure may be structurally and chemically characterized as described below and in the following Examples. The amino acid sequence ID numbers of the heavy/light chain variable regions of the antibodies are summarized in Table 1 below, some antibodies sharing the same V_{H} or V_{L}. The heavy chain constant region for the antibodies may be human IgGl heavy chain constant region having the amino acid sequence set forth in, e.g., SEQ ID NOs: 12 or 57, or human IgG4 heavy chain constant region having the amino acid sequence set forth in, e.g., SEQ ID NO: 13, and the light chain constant region for the antibodies may be human kappa constant region having the amino acid sequence set forth in, e.g., SEQ ID NO: 14. These antibodies may also contain mouse IgGl or IgG2 heavy chain constant region, and/or mouse kappa constant region. The antibody may consist of two heavy chain and two light chains connected by disulfide bonds, the C-terminus of the heavy chain variable region is linked to the N-terminus of the heavy chain constant region, and the C-terminus of the light chain variable region is linked to the N-terminus of the light chain constant region.

The heavy chain variable region CDRs and the light chain variable region CDRs in Table 1 have been defined by the Kabat numbering system. However, as is well known in the art, CDR regions can also be determined by other systems such as Chothia, IMGT, AbM, or Contact numbering system/method, based on heavy chain/light chain variable region sequences.

The V_{H} and V_{L} sequences (or CDR sequences) of other anti-TSLP antibodies which bind to human TSLP can be “mixed and matched” with the V_{H} and V_{L} sequences (or CDR sequences) of the anti-TSLP antibody of the present disclosure. Preferably, when V_{H} and V_{H} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one embodiment, an antibody of the disclosure, or an antigen binding portion thereof, comprises:
(a) a heavy chain variable region comprising an amino acid sequence listed above in Table 1; and
(b) a light chain variable region comprising an amino acid sequence listed above in Table 1, or the V_{L} of another anti-TSLP antibody, wherein the antibody specifically binds human TSLP.

TABLE 1
Amino acid sequence ID numbers of heavy/light chain variable regions

| | Heavy chain | | | | Light chain | | | |
|---|---|---|---|---|---|---|---|---|
| Antibody | V_{H} CDR1 | V_{H} CDR2 | V_{H} CDR3 | V_{H} | V_{L} CDR1 | V_{L} CDR2 | V_{L} CDR3 | V_{L} |
| Mouse/chimeric | SEQ ID | SEQ ID | SEQ ID | SEQ ID NO.: 7 | SEQ ID | SEQ ID | SEQ ID | SEQ ID NO.: 10 |
| 1C5F12E9 | NO.: 1 | NO.: 2 | NO.: 3 | | NO.: 4 | NO.: 5 | NO.: 6 | |
| hu1C5F12E9-V1 | | | | SEQ ID NO.: 8 | | | | SEQ ID NO: 11, X1 = S, |
| | | | | | | | | X2 = V |
| hu1C5F12E9-V2 | | | | SEQ ID NO.: 9, X1 = R, | | | | SEQ ID NO: 11, X1 = S, |
| | | | | X2 = V, X3 = R | | | | X2 = V |
| hu1C5F12E9-V3 | | | | SEQ ID NO: 9, X1 = R, | | | | SEQ ID NO: 11, X1 = S, |
| | | | | X2 = V, X3 = V | | | | X2 = V |
| hu1C5F12E9-V4 | | | | SEQ ID NO: 9, X1 = R, | | | | SEQ ID NO: 11, X1 = S, |
| | | | | X2 = A, X3 = R | | | | X2 = V |
| hu1C5F12E9-V5 | | | | SEQ ID NO: 9, X1 = K, | | | | SEQ ID NO: 11, X1 = S, |
| | | | | X2 = A, X3 = R | | | | X2 = V |
| hu1C5F12E9-V6 | | | | SEQ ID NO: 9, X1 = K, | | | | SEQ ID NO: 11, X1 = S, |
| | | | | X2 = A, X3 = V | | | | X2 = V |
| hu1C5F12E9-V7 | | | | SEQ ID NO.: 8 | | | | SEQ ID NO: 11, X1 = A, |
| | | | | | | | | X2 = I |
| hu1C5F12E9-V8 | | | | SEQ ID NO.: 9, X1 = R, | | | | SEQ ID NO: 11, X1 = A, |
| | | | | X2 = V, X3 = R | | | | X2 = I |
| hu1C5F12E9-V9 | | | | SEQ ID NO: 9, X1 = R, | | | | SEQ ID NO: 11, X1 = A, |
| | | | | X2 = V, X3 = V | | | | X2 = I |
| hu1C5F12E9-V10 | | | | SEQ ID NO: 9, X1 = R, | | | | SEQ ID NO: 11, X1 = A, |
| | | | | X2 = A, X3 = R | | | | X2 = I |
| hu1C5F12E9-V11 | | | | SEQ ID NO: 9, X1 = K, | | | | SEQ ID NO: 11, X1 = A, |
| | | | | X2 = A, X3 = R | | | | X2 = I |
| hu1C5F12E9-V12 | | | | SEQ ID NO: 9, X1 = K, | | | | SEQ ID NO: 11, X1 = A, |
| | | | | X2 = A, X3 = V | | | | X2 = I |
| hu1C5F12E9-V13 | | | | SEQ ID NO.: 8 | | | | SEQ ID NO: 11, X1 = S, |
| | | | | | | | | X2 = I |
| hu1C5F12E9-V14 | | | | SEQ ID NO.: 9, X1 = R, | | | | SEQ ID NO: 11, X1 = S, |
| | | | | X2 = V, X3 = R | | | | X2 = I |
| hu1C5F12E9-V15 | | | | SEQ ID NO: 9, X1 = R, | | | | SEQ ID NO: 11, X1 = S, |
| | | | | X2 = V, X3 = V | | | | X2 = I |
| hu1C5F12E9-V16 | | | | SEQ ID NO: 9, X1 = R, | | | | SEQ ID NO: 11, X1 = S, |
| | | | | X2 = A, X3 = R | | | | X2 = I |
| hu1C5F12E9-V17 | | | | SEQ ID NO: 9, X1 = K, | | | | SEQ ID NO: 11, X1 = S, |
| | | | | X2 = A, X3 = R | | | | X2 = I |
| hu1C5F12E9-V18 | | | | SEQ ID NO: 9, X1 = K, | | | | SEQ ID NO: 11, X1 = S, |
| | | | | X2 = A, X3 = V | | | | X2 = I |
| Mouse | SEQ ID | SEQ ID | SEQ ID | SEQ ID NO.: 43 | SEQ ID | SEQ ID | SEQ ID | SEQ ID NO.: 44 |
| D1D8H9F7 | NO.: 37 | NO.: 38 | NO.: 39 | | NO.: 40 | NO.: 41 | NO.: 42 | |
| Mouse | SEQ ID | SEQ ID | SEQ ID | SEQ ID NO.: 53 | SEQ ID | SEQ ID | SEQ ID | SEQ ID NO.: 54 |
| D1C2H1H1 | NO.: 47 | NO.: 48 | NO.: 49 | | NO.: 50 | NO.: 51 | NO.: 52 | |

In another embodiment, an antibody of the disclosure, or an antigen binding portion thereof, comprises:
(a) the CDR1, CDR2, and CDR3 regions of the heavy chain variable region listed above in Table 1; and
(b) the CDR1, CDR2, and CDR3 regions of the light chain variable region listed above in Table 1 or the CDRs of another anti-TSLP antibody, wherein the antibody specifically binds human TSLP.

In yet another embodiment, the antibody, or antigen binding portion thereof, includes the heavy chain variable CDR2 region of anti-TSLP antibody combined with CDRs of other antibodies which bind human TSLP, e.g., CDR1 and/or CDR3 from the heavy chain variable region, and/or CDR1, CDR2, and/or CDR3 from the light chain variable region of a different anti-TSLP antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116:2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8: Scientific Review 8 (2001); Igarashi et al., J. Biochem (Tokyo) 117:452-7 (1995); Bourgeois et al., J. Virol 72:807-10 (1998); Levi et aL, Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these references is hereby incorporated by reference in its entirety.

Accordingly, in another embodiment, antibodies of the disclosure comprise the CDR2 of the heavy chain variable region of the anti-TSLP antibody and at least the CDR3 of the heavy and/or light chain variable region of the anti-TSLP antibody, or the CDR3 of the heavy and/or light chain variable region of another anti-TSLP antibody, wherein the antibody is capable of specifically binding to human TSLP. These antibodies preferably (a) compete for binding with TSLP; (b) retain the functional characteristics; (c) bind to the same epitope; and/or (d) have a similar binding affinity as the anti-TSLP antibody of the present disclosure. In yet another embodiment, the antibodies further may comprise the CDR2 of the light chain variable region of the anti-TSLP antibody, or the CDR2 of the light chain variable region of another anti-TSLP antibody, wherein the antibody is capable of specifically binding to human TSLP. In another embodiment, the antibodies of the disclosure further may include the CDR1 of the heavy and/or light chain variable region of the anti-TSLP antibody, or the CDR1 of the heavy and/or light chain variable region of another anti-TSLP antibody, wherein the antibody is capable of specifically binding to human TSLP.

### Conservative Modifications

In another embodiment, an antibody of the disclosure comprises a heavy and/or light chain variable region sequences of CDR1, CDR2 and CDR3 sequences which differ from those of the anti-TSLP antibodies of the present disclosure by one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J. Biol. Chem. 272:26864-26870; Hall et al., (1992) J Immunol. 149:1605-12; Kelley and O'Connell (1993) Biochem. 32:6862-35; Adib-Conquy et al., (1998) Int. Immunol. 10:341-6 and Beers et al., (2000) Clin. Can. Res. 6:2835-43.

Accordingly, in one embodiment, the antibody comprises a heavy chain variable region comprising CDR1, CDR2, and CDR3 sequences and/or a light chain variable region comprising CDR1, CDR2, and CDR3 sequences, wherein:
(a) the heavy chain variable region CDR1 sequence comprises a sequence listed in Table 1 above, and/or conservative modifications thereof, and/or
(b) the heavy chain variable region CDR2 sequence comprises a sequence listed in Table 1 above, and/or conservative modifications thereof, and/or
(c) the heavy chain variable region CDR3 sequence comprises a sequence listed in Table 1 above, and/or conservative modifications thereof, and/or
(d) the light chain variable region CDR1, and/or CDR2, and/or CDR3 sequences comprise the sequence(s) listed in Table 1 above; and/or conservative modifications thereof; and
(e) the antibody specifically binds human TSLP.

The antibody of the present disclosure possesses one or more of the following functional properties described above, such as high affinity binding to human TSLP.

In various embodiments, the antibody can be, for example, a mouse, human, humanized or chimeric antibody.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

### Engineered and Modified Antibodies

Antibodies of the disclosure can be prepared using an antibody having one or more of the V_{H}/V_{L} sequences of the anti-TSLP antibody of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the disclosure pertains to an isolated monoclonal antibody, or antigen binding portion thereof, comprising a heavy chain variable region comprising CDR1, CDR2, and CDR3 sequences comprising the sequences of the present disclosure, as described above, and/or a light chain variable region comprising CDR1, CDR2, and CDR3 sequences comprising the sequences of the present disclosure, as described above. While these antibodies contain the V_{H} and V_{L} CDR sequences of the monoclonal antibody of the present disclosure, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG-0010109, NT-024637 & BC070333), 3-33 (NG-0010109 & NT-024637) and 3-7 (NG-0010109 & NT-024637). As another example, the following heavy chain germline sequences found in the HCol2 HuMAb mouse are available in the accompanying Genbank Accession Nos.: 1-69 (NG-0010109, NT-024637 & BC070333), 5-51 (NG-0010109 & NT-024637), 4-34 (NG-0010109 & NT-024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the disclosure are those that are structurally similar to the framework sequences used by antibodies of the disclosure. The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Accordingly, in another embodiment, the disclosure provides isolated anti-TSLP monoclonal antibodies, or antigen binding portions thereof, comprising a heavy chain variable region comprising: (a) a V_{H} CDR1 region comprising the sequence of the present disclosure, or an amino acid sequence having one, two, three, four or five amino acid substitutions, deletions or additions; (b) a V_{H} CDR2 region comprising the sequence of the present disclosure, or an amino acid sequence having one, two, three, four or five amino acid substitutions, deletions or additions; (c) a V_{H} CDR3 region comprising the sequence of the present disclosure, or an amino acid sequence having one, two, three, four or five amino acid substitutions, deletions or additions; (d) a V_{L} CDR1 region comprising the sequence of the present disclosure, or an amino acid sequence having one, two, three, four or five amino acid substitutions, deletions or additions; (e) a V_{L} CDR2 region comprising the sequence of the present disclosure, or an amino acid sequence having one, two, three, four or five amino acid substitutions, deletions or additions; and (f) a V_{L} CDR3 region comprising the sequence of the present disclosure, or an amino acid sequence having one, two, three, four or five amino acid substitutions, deletions or additions.

Engineered antibodies of the disclosure include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689 B2.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure can be chemically modified (e.g., one or more chemical moieties can be attached to the antibody) or be modified to alter its glycosylation, again to alter one or more functional properties of the antibody.

In one embodiment, the C_{H1}-hinge region is modified in such that the number of cysteine residues in the hinge region is altered, e.g., increased or decreased. This approach is described further in U.S. Pat. No. 5,677,425. The number of cysteine residues in the C_{H1}-hinge region is altered to, for example, facilitate assembly of the light and heavy chains or to increase or decrease the stability of the antibody.

In another embodiment, the Fchinge region of an antibody is mutated to decrease the biological half-life of the antibody. More specifically, one or more amino acid mutations are introduced into the C_{H}2-C_{H}3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (a (1, 6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the a-1, 6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lecl3 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. Methods for production of antibodies in a plant system are disclosed in the U.S. patent application corresponding to Alston & Bird LLP attorney docket No. 040989/314911, filed on Aug. 11, 2006. PCT Publication WO 99/54342 describes cell lines engineered to express glycoprotein-modifying glycosyl transferases (e.g., 0(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180). Alternatively, the fucose residues of the antibody can be cleaved off using a fucosidase enzyme; e.g., the fucosidase a-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (Ci-Cio) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EPO 154 316 and EP 0 401 384.

### Antibody's Physical Properties

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence. In some instances, it is preferred to have an anti-TSLP antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a link into the polypeptide chain and decreases its stability (isoaspartic acid effect).

Each antibody will have a unique isoelectric point (pI), which generally falls in the pH range between 6 and 9.5. The pI for an IgGl antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-TSLP antibody that contains a pI value that falls in the normal range. This can be achieved either by selecting antibodies with a pI in the normal range or by mutating charged surface residues.

### Nucleic Acid Molecules Encoding Antibodies of the Disclosure

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and V_{L} sequences of the TSLP monoclonal antibody or the CDRs. Once DNA fragments encoding V_{H} and VL segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgGl, IgG2, IgG3, IgG4, IgA, IgE, IgM or IgD constant region, but most preferably is an IgGl or IgG4 constant region. For a Fab fragment heavy chain gene, the V_{H}-encoding DNA can be operatively linked to another DNA molecule encoding only the heavy chain C_{HI} constant region.

The isolated DNA encoding the V_{L} region can be converted to a frill-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

### Production of Monoclonal Antibodies of the Disclosure

Monoclonal antibodies (mAbs) of the present disclosure can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

### Generation of Transfectomas Producing Monoclonal Antibodies of the Disclosure

Antibodies of the disclosure also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRa promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr-CHO cells, described in Urlaub and Chasin, (1980)Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Immunoconjugates

Antibodies of the disclosure can be conjugated to a therapeutic agent to form an immunoconjugate such as an antibody-drug conjugate (ADC). Suitable therapeutic agents include cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Pat. Nos. 7,691,962; 7,517,903; and 7,714,016; the disclosures of which are incorporated herein by reference.

### Bispecific Molecules

In another aspect, the present disclosure features bispecific molecules comprising one or more antibodies of the disclosure linked to at least one other functional molecule, e.g., another peptide or protein (e.g., another antibody or ligand for a receptor) to generate a bispecific molecule that binds to at least two different binding sites or target molecules. Thus, as used herein, “bispecific molecule” includes molecules that have three or more specificities.

In an embodiment, a bispecific molecule has, in addition to an anti-Fc binding specificity and an anti-TSLP binding specificity, a third specificity. The third specificity can be for IgE, IL4, IL4R, IL13, IL13R, IL-5, IL5R or TSLPR/IL7R for asthma treatment.

Bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv)2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

### Antibody-Encoding or Antibody-Bearing Oncolytic Virus

An oncolytic virus preferentially infects and kills cancer cells. Antibodies of the present disclosure can be used in conjunction with oncolytic viruses. Alternatively, oncolytic viruses encoding antibodies of the present invention can be introduced into human body.

### Chimeric Antigen Receptor

Also provided herein are a chimeric antigen receptor (CAR) containing an anti-TSLP scFv, the anti-TSLP scFv comprising CDRs and heavy/light chain variable regions described herein.

The anti-TSLP CAR may comprise (a) an extracellular antigen binding domain comprising an anti-TSLP scFv; (b) a transmembrane domain; and (c) an intracellular signaling domain.

The CAR may contain a signal peptide at the N-terminus of the extracellular antigen binding domain that directs the nascent receptor into the endoplasmic reticulum, and a hinge peptide at the N-terminus of the extracellular antigen binding domain that makes the receptor more available for binding. The CAR preferably comprises, at the intracellular signaling domain, a primary intracellular signaling domain and one or more co-stimulatory signaling domains. The mainly used and most effective primary intracellular signaling domain is CD3-zeta cytoplasmic domain which contains ITAMs, the phosphorylation of which results in T cell activation. The co-stimulatory signaling domain may be derived from the co-stimulatory proteins such as CD28, CD137 and OX40.

The CARs may further add factors that enhance T cell expansion, persistence, and anti-tumor activity, such as cytokines, and co-stimulatory ligands.

Also provided are engineered immune effector cells, comprising the CAR provided herein. In some embodiments, the immune effector cell is a T cell, an NK cell, a peripheral blood mononuclear cell (PBMC), a hematopoietic stem cell, a pluripotent stem cell, or an embryonic stem cell. In some embodiments, the immune effector cell is a T cell.

### Pharmaceutical Compositions

In another aspect, the present disclosure provides a pharmaceutical composition comprising one or more antibodies (or antigen-binding portion thereof, the bispecifics, immunoconjugates, CAR-immune cells, antibody-encoding or antibody-bearing oncolytic viruses, or antibody-encoding nucleic acid molecules) of the present disclosure formulated together with a pharmaceutically acceptable carrier. The antibodies (or antigen-binding portion thereof, or the bispecifics) can be dosed sperapartely when the composition contains more than one antibody (or antigen-binding portion thereof, or the bispecifics). The composition may optionally contain one or more additional pharmaceutically active ingredients, such as another antibody or a drug, such as an anti-asthma drug, an anti-ulcerative colitis drug, an anti-atopic dermatitis drug, or an anti-psoriasis drug.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from e.g., about 0.0001 to 100 mg/kg of the host body weight. The treatment regime may comprise administration e.g., once per week.

A “therapeutically effective dosage” of an anti-TSLP antibody, or the antigen-binding portion thereof, or the bispecifics, CAR-immune cells, oncolytic viruses, immunoconjugates of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic antibody can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol.29:685; Umezawa et al., (1988) Biochem. Biophys. Res. Commun. 153:1038; Bloeman et al., (1995) FEBS Lett.357:140; M. Owais et al., (1995) Antimicrob. Agents Chemother. 39:180; Briscoe et al., (1995) Am. J. Physiol. 1233:134; Schreier et al., (1994) J Biol. Chem. 269:9090; Keinanen and Laukkanen (1994) FEBS Lett. 346:123; and Killion and Fidler (1994) Immunomethods 4:273.

### Uses and Methods of the Disclosure

The composition comprising the antibodies or the antigen-binding portion thereof, or the bispecific molecules, immunoconjugates, CAR-immune cells, antibody-encoding or antibody-bearing oncolytic viruses, or antibody-encoding nucleic acid molecules of the present disclosure have numerous in vitro and in vivo utilities involving, for example, treatment of asthma, ulcerative colitis, atopic dermatitis or psoriasis. The antibodies can be administered to human subjects, e.g., in vivo, to alleviate these diseases.

In another aspect, the disclosure provides methods of combination therapy in which the anti-TSLP antibodies, or antigen-binding portion thereof, or the bispecifics, immunoconjugates, CAR-immune cells, antibody-encoding or antibody-bearing oncolytic viruses of the present disclosure are co-administered with one or more additional antibodies that are effective in alleviate asthma, ulcerative colitis, atopic dermatitis or psoriasis in a subject. In one embodiment, the disclosure provides a method for treating asthma in a subject comprising administering to the subject an anti-TSLP antibody (or antigen-binding portion thereof, the specific antibody, the immunoconjugate, the CAR-immune cell, the antibody-encoding or antibody-bearing oncolytic virus, or the antibody-encoding nucleic acid molecule) and one or more additional antibodies, such as an anti-TSLPR antibody, an anti-IL4 antibody, an anti-IL4R antibody, an anti-IL13 antibody, an anti-IL13R antibody, an anti-IL-5 antibody, an anti-IL5R antibody, and/or an anti-IgE antibody. In certain embodiments, the subject is human.

The TSLP signaling blocking can also be further combined with standard disease treatments, such as asthma treatment. For example, TSLP signaling blocking can be combined with administration of the antibodies mentioned above for asthma treatment, and reduction or avoidance of exposure to allergens.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

The present disclosure is further illustrated by the following examples, which should not be construed as further limiting. The contents of all figures and all references, Genbank sequences, patents and published patent applications cited throughout this application are expressly incorporated herein by reference.

### Example 1 Generation of Mouse Anti-TSLP Monoclonal Antibodies Using Hybridoma Technology

### Immunization

Mice were immunized according to the method as described in E Harlow, D. Lane, Antibody: A Laboratory Manual, Cold Spring Harbor Laboratory Press, Cold Spring Harbor, N.Y., 1998. Recombinant human TSLP protein (Biosion Inc., amino acid residue 21-159 of Uniprot No. Q969D9) with human IgGl Fc tag at the C-terminus was used as the immunogen. Human TSLP-his protein (Biosion Inc., Cat #P100273) was used for determining anti-sera titer and for screening hybridomas secreting antigen-specific antibodies. Immunizing dosages contained 25 μg human TSLP-Fc protein/mouse/injection for both primary and boost immunizations. To increase immune response, the complete Freud's adjuvant and incomplete Freud's adjuvant (Sigma®, St. Louis, Mo., USA) were used respectively for primary and boost immunizations. Briefly, adjuvant-antigen mixture was prepared by first gently mixing the adjuvant in a vial using a vortex. The desired amount of adjuvant was transferred to an autoclaved 1.5 mL micro-centrifuge tube. The antigen was prepared in PBS or saline with concentrations ranging from 0.25-0.34 mg/ml. The calculated amount of antigen was then added to the micro-centrifuge tube with the adjuvant, and the resulting mixture was mixed by gently vortexing for 2 minutes to generate water-in-oil emulsions. The adjuvant-antigen emulsion was then drawn into the proper syringe for animal injection. A total of 25 μg of antigen was injected in a volume of 150-200 μl. Each animal was immunized, and then boosted for 3 to 4 times depending on the anti-sera titer. Animals with good titers were given a final boost by intraperitoneal injection before fusion.

### Hybridoma Fusion and Screening

Cells of murine myeloma cell line (SP2/0-Agl4, ATCC #CRL-1581) were cultured to reach the log phase stage right before fusion. Spleen cells from immunized mice were prepared sterilely and fused with myeloma cells according to the method as described in Kohler G, and Milstein C, “Continuous cultures of fused cells secreting antibody of predefined specificity,” Nature, 256: 495-497(1975). Fused “hybrid cells” were subsequently dispensed into 96-well plates in DMEM/20% FCS/HAT media. Surviving hybridoma colonies were observed under the microscope seven to ten days post fusion. After two weeks, the supernatant from each well was subject to Capture ELISA using recombinant human TSLP-his protein. Positive hybridoma clones producing antibodies that showed high specific TSLP binding activities were subcloned by limited dilution to ensure the clonality of the cell line, and then the generated monoclonal antibodies were purified. Briefly, Protein A sepharose column (from Bestchrom (Shanghai) Biosciences, Cat #AA0273) was washed using PBS buffer in 5 to 10 column volumes. Cell supernatants were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing immunoglobulins were pooled and dialyzed in PBS overnight at 4° C. Subsequently, the in vitro functional activities of purified monoclonal antibodies were characterized as follows.

### Example 2 Affinity Determination of Exemplary Mouse Anti-TSLP Monoclonal Antibodies Using BIACORE® Surface Plasmon Resonance

The purified anti-TSLP mouse monoclonal antibodies (mAbs) generated in Example 1 were characterized for the binding affinities and binding kinetics by Biacore® T200 system (GE Healthcare™, Pittsburgh, PA, USA).

Briefly, Biosion® in house synthesized recombinant human TSLP-his (SEQ ID NO.: 28) or cynomolgus monkey TSLP-his protein (SEQ ID NO.: 29) in CH₃COONa buffer (provided by Biocore®) at the concentration of 10 μg/mL was covalently linked to a CM5 chip (carboxy methyl dextran coated chip from GE Healthcare™ #BR100530) via primary amines, using a standard amine coupling kit provided by Biacore (GE Healthcare™, Pittsburgh, PA, USA). Un-reacted moieties on the biosensor surface were blocked with ethanolamine. Then, serially diluted purified anti-TSLP antibodies (2-fold serial dilution in HBS-EP⁺ buffer, starting at 100 nM) and Tezepelumab as a benchmark (also referred to as TSLP-BM, prepared in-house using the heavy chain and light chain amino acids set forth in SEQ ID NOs.: 35 and 36, 2-fold serial dilution in HBS-EP⁺ buffer starting at 100 nM) were respectively flowed onto the chip at a flow rate of 50 μL/min. The antigen-antibody association kinetics was followed for 4 minutes and the dissociation kinetics was followed for 13 minutes. The association and dissociation curves were fit to a 1:1 Langmuir binding model using BIAcore® evaluation software, and the K_{D}, Kₐ and K_{d} values were determined and shown in Table 2 below.

TABLE 2
Binding affinities of mouse anti-TSLP antibodies

| | Kinetics on Biacore ® | | | | | |
|---|---|---|---|---|---|---|
| | Human TSLP | | | cynomolgus TSLP | | |
| | Kₐ | K_{d} | K_{D} | Kₐ | K_{d} | K_{D} |
| Mouse mAb | (M⁻¹s⁻¹) | (s⁻¹) | (M) | (M⁻¹s⁻¹) | (S⁻¹) | (M) |
| 1F5H12D7 | 8.97E+04 | <1.00E−05 | <1.12E−10 | not tested | | |
| 1G3B9B1 | 9.36E+04 | <1.00E−05 | <1.07E−10 | not tested | | |
| 1G2H12A1 | 8.52E+04 | <1.00E−05 | <1.17E−10 | not tested | | |
| 1H3F11B7 | 9.29E+04 | <1.00E−05 | <1.08E−10 | not tested | | |
| 1B5D5A5 | not tested | | | not tested | | |
| 1C5F12E9 | 9.11E+04 | <1.00E−05 | <1.10E−10 | not tested | | |
| Tezepelumab | 2.25E+05 | 3.04E−05 | 1.35E−10 | not tested | | |
| D1D8H9F7 | 1.72E+05 | 3.09E−05 | 1.80E−10 | No-binding | | |
| D1C2H1H1 | 6.72E+05 | <1.00E−05 | <1.49E−11 | / | / | 7.23E−09 |
| Tezepelumab | 5.00E+05 | 5.57E−05 | 1.12E−10 | 6.63E+05 | 4.86E−05 | 7.33E−11 |

All the tested anti-TSLP antibodies specifically bound to human TSLP, with most having higher binding affinities than the benchmark. The antibody D1C2H1H1 also showed binding affinity to monkey TSLP.

### Example 3 Binding Capacities of Exemplary Mouse Anti-TSLP Monoclonal Antibodies

The binding activities of all the mouse anti-TSLP antibodies were determined by Capture ELISA and indirect ELISA.

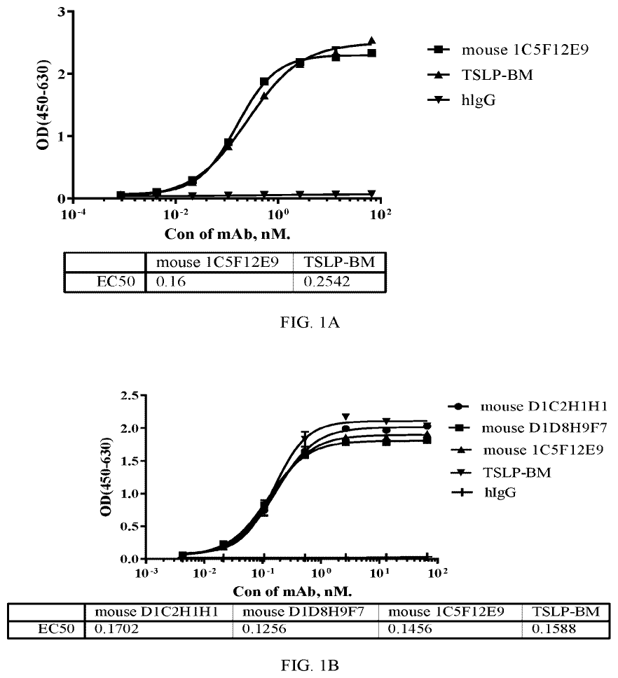

For the capture ELISA, 96-well micro plates were coated with 100 μl of 2 μg/ml AffiniPure® goat anti-mouse IgG Fey fragment specific (Jackson Immuno Research, Cat #115-005-008) in PBS and incubated overnight at 4° C. Plates were washed 4 times with wash buffer (PBS+0.05% Tween-20, PBST) and then blocked with 200 μl/well blocking buffer (5% w/v non-fatty milk in PBST) for 2 hours at 37° C. Plates were washed again and incubated with 100 μl of serially diluted anti-TSLP antibodies of the present disclosure, Tezepelumab or hIgG (Hualan Biological Engineering Inc.), 5-fold serial dilution in 2.5% non-fatty milk in PBST starting at 66.7 nM, for 40 minutes at 37° C., and then washed 4 times again. Plates containing captured antibodies were incubated with 100 μl biotin-labeled human TSLP-his proteins (SEQ ID NO.: 28, prepared in-house, 0.23 nM in 2.5% non-fatty milk in PBST) for 40 minutes at 37° C., washed 4 times, and incubated with streptavidin conjugated HRP (1:10000 dilution in PBST, Jackson Immuno Research, Cat #016-030-084, 100 μl/well) for 40 minutes at 37° C. After the final wash, plates were incubated with 100 μl/well TMB (Innoreagents). The reaction was stopped 15 minutes later at room temperature with 50 μl/well 1M H₂SO₄, and the absorbance of each well was read on a microplate reader using dual wavelength mode with 450 nm for TMB and 630 nm as the reference wavelength. The OD (450-630) values were plotted against antibody concentration. Data was analyzed using Graphpad Prism® software and EC₅₀ values were reported. The results of some antibodies were shown in FIGS. 1A-1B.

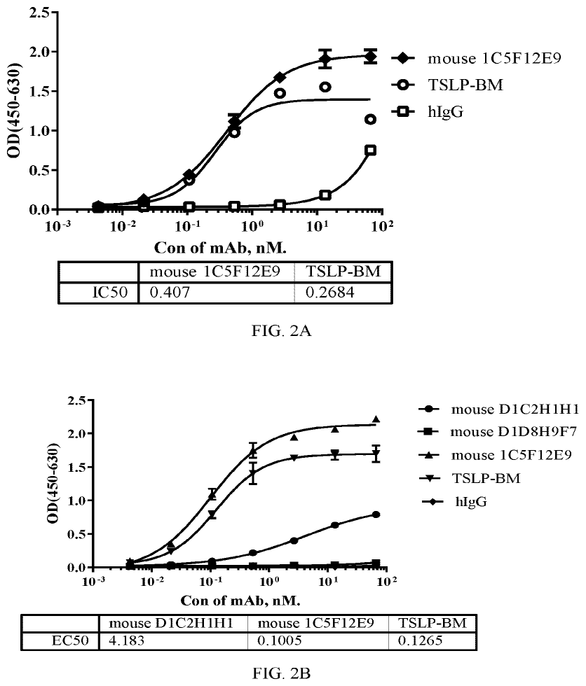

For the indirect ELISA, 96-well micro plates were coated with 100 μl of 2 μg/ml cynomolgus TSLP-his protein (SEQ ID NO.: 29, prepared in-house) in carbonate/bicarbonate buffer (pH 9.6) overnight at 4° C. ELISA plates were washed 4 times with wash buffer (PBS+0.05% Tween-20, PBST) and then blocked with 200 μl/well blocking buffer (5% w/v non-fatty milk in PBST) for 2 hours at 37° C. Plates were washed again and incubated with 100 μl of serially diluted anti-TSLP antibodies of the disclosure, Tezepelumab or hIgG (5-fold serial dilution in 2.5% non-fatty milk in PBST starting at 66.7 nM) for 40 minutes at 37° C. ELISA plates were washed 4 times again and incubated with Peroxidase AffiniPure® F(ab′)₂ Fragment Goat Anti-Mouse IgG, Fey Fragment Specific (1:5000 dilution in PBST buffer, Jackson Immunoresearch, Cat #115-036-071, 100 μl/well) for 40 minutes at 37° C. After the final wash, plates were incubated with 100 μl/well TMB (Innoreagents). The reaction was stopped 15 minutes later at room temperature with 50 μl/well 1M H₂SO₄, and the absorbance of each well was read on a microplate reader using dual wavelength mode with 450 nm for TMB and 630 nm as the reference wavelength. The OD (450-630) values were plotted against antibody concentration. Data was analyzed using Graphpad Prism® software and EC₅₀ values were reported. The results of some antibodies were shown in FIGS. 2A-2B.

Data showed that most antibodies of the disclosure specifically bound to human and monkey TSLPs. The Emax (maximal binding) and EC₅₀ of 1C5F12E9, D1D8H9F7 and D1C2H1H1 were similar to those of the benchmark, as shown in FIGS. 1A-1B, wherein D1D8H9F7 had lower EC₅₀, suggesting it bound to human TSLP more efficiently. It can be seen from FIGS. 2A-2B that 1C5F12E showed higher Emax in monkey TSLP binding assay as compared to the benchmark.

### Example 4 Functional Assays on Anti-TSLP Antibodies

### 4.1 Ligand Blocking ELISA

The abilities of all the anti-TSLP antibodies to block TSLP-TSLPR/IL7R binding were measured using a competitive ELISA assay. Briefly, 100 μl human TSLPR-Fc proteins (SEQ ID NO.: 30, prepared in-house) at 1 μg/mL in PBS, and 100 μl human IL7Ra-Fc proteins (SEQ ID NO.: 31, prepared in-house) at 1 μg/mL in PBS were coated on 96-well micro plates overnight at 4° C. The next day, plates were washed with wash buffer (PBS+0.05% Tween-20, PBST), and blocked with 5% w/v non-fatty milk in PBST for 2 hours at 37° C. Plates were then washed again using wash buffer.

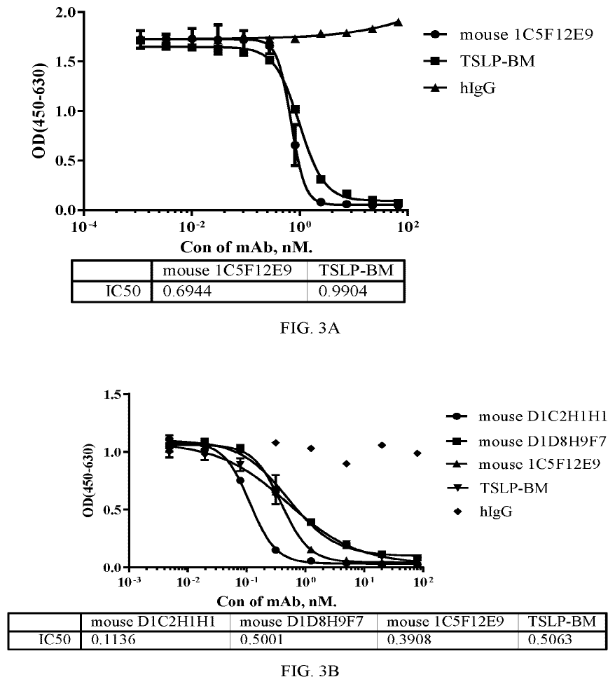

The anti-TSLP antibodies or controls were diluted in biotin-labeled human TSLP-Fc (SEQ ID NO.: 32, prepared in-house, 0.29 nM in 2.5% non-fatty milk in PBST), starting at 66.7 nM with a 3-fold serial dilution, and incubated at room temperature for 40 minutes. Then 100 μl of the antibody/TSLP-Fc mixtures were added to TSLPR/IL7R-coated plates. After incubation at 37° C. for 40 minutes, plates were washed 4 times using wash buffer. Then streptavidin conjugated HRP was added and incubated for 40 minutes at 37° C. to detect biotin-labeled human TSLP-Fc bound to TSLPR/IL7R. Plates were washed again using wash buffer. Finally, TMB was added and the reaction was stopped using 1M H₂SO₄. The absorbance of each well was read on a microplate reader using dual wavelength mode with 450 nm for TMB and 630 nm as the reference wavelength, then the OD (450-630) values were plotted against antibody concentration. Data was analyzed using Graphpad Prism® software and IC₅₀ values were reported. The results of some antibodies were shown in FIGS. 3A-3B.

### 4.2 Benchmark Blocking ELISA

The abilities of the anti-TSLP antibodies to block Benchmark (Tezepelumab)-human TSLP binding were measured using a competitive ELISA assay. Briefly, 2 μg/mL Tezepelumab in PBS was coated on 96-well micro plates, 100 μl/well, and incubated overnight at 4° C. The next day, plates were washed with wash buffer (PBS+0.05% Tween-20, PBST), and blocked with 5% w/v non-fatty milk in PBST for 2 hours at 37° C.

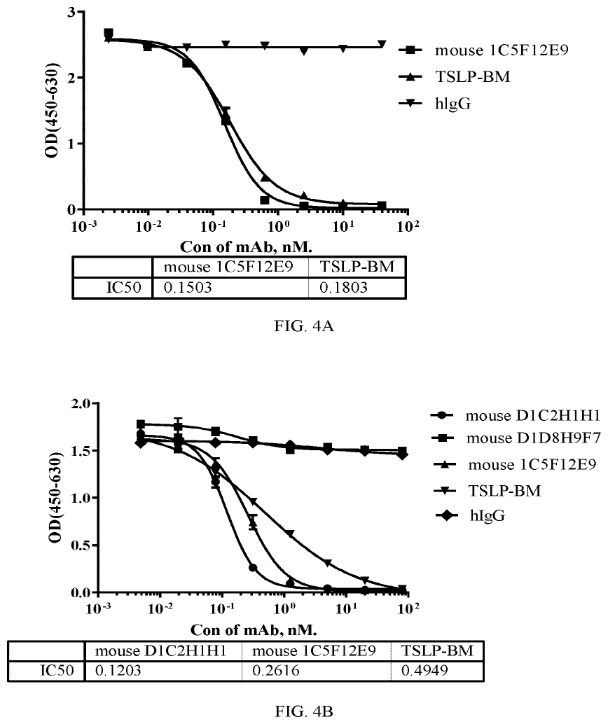

Meanwhile, anti-TSLP antibodies or controls were diluted in biotin-labeled human TSLP-Fc (SEQ ID NO.: 32, 0.047 nM in 2.5% non-fatty milk in PBST), starting at 40 nM with a 4-fold serial dilution, and incubated at room temperature for 40 minutes. Then, 100 μl of the antibody/TSLP-Fc-biotin mixtures were added to Benchmark-coated plates. After incubation at 37° C. for 40 minutes, plates were washed 4 times using wash buffer. Then streptavidin conjugated HRP was added, and the plates were incubated for 40 minutes at 3 7° C. to detect biotin-labeled human TSLP-Fc bound to Benchmark. Plates were finally washed using wash buffer. TMB was added and the reaction was stopped using 1M H₂SO₄. The absorbance of each well was read on a microplate reader using dual wavelength mode with 450 run for TMB and 630 nm as the reference wavelength, and the OD (450-630) values were plotted against antibody concentration. Data was analyzed using Graphpad Prism® software and IC₅₀ values were reported. The results of some antibodies were shown in FIGS. 4A-4B.

### 4.3 Cell-Based Ligand-Blocking FACS

The activities of anti-TSLP antibodies to block the binding of TSLP-Fc protein to cell surface TSLPR/IL7R were evaluated in a Flow Cytometry (FACS) assay, using a cell line BAF3-3E6 expressing cell-surface human TSLPR (amino acid residues 1-371 of uniprot No. Q9HC73.1, SEQ ID NO.: 33) and human IL7R (amino acid residues 1-459 of uniprot No. P16871.1, SEQ ID NO.: 34). The BAF3-3E6 cell line was prepared following the instruction of lipofectamine 3000 transfection reagent (Thermo Fisher), by transfecting BAF3 cells (iCell Bioscience Inc., Cat #MIMCL-021) with pCMV-T-P plasmids inserted with TSLPR coding sequence between EcoRI and Xbal sites and pCMV3-SP plasmids inserted with IL7R coding sequence between HindIII and Xbal sites.

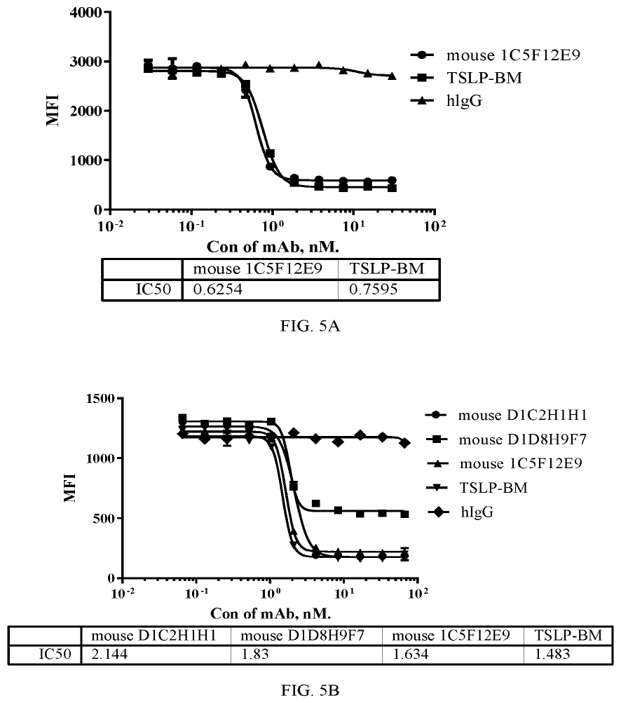

Briefly, the anti-TSLP antibodies of the disclosure, the benchmark or negative control hIgG (human immunoglobulin (pH4) for intravenous injection, Hualan Biological Engineering Inc.) were diluted with human TSLP-Fc solution (SEQ ID NO.: 32, prepared in-house, 0.38 nM in FACS buffer), 2-fold serial dilution starting at 30 nM, and incubated at room temperature for 40 minutes. BAF3-3E6 cells were harvested from cell culture flasks, washed twice and re-suspended in phosphate buffered saline (PBS) containing 2% v/v Fetal Bovine Serum (FACS buffer). 1×10⁵ cells per well in 96 well-plates were incubated with 100 μl/well antibody/TSLP-Fc-biotin mixtures for 40 minutes at 4° C. Cells were washed twice with FACS buffer, and then added and incubated with 100 μl/well R-Phycoerythrin Streptavidin (1:1000 dilution in FACS buffer, Jackson Immunoresearch, Cat #016-110-084) for 40 minutes at 4° C. in dark. Cells were washed twice and re-suspended in FACS buffer. Fluorescence was measured using a Becton Dickinson® FACS Canto II-HTS equipment. Data was analyzed using Graphpad Prism® software and IC₅₀ values were reported. The results of some antibodies were shown in FIGS. 5A-5B.

### 4.4 Cell-Based Functional Assay

BAF3 cell proliferation and survival is normally dependent on IL-3. However, when these cells are engineered to express human TSLPR and human IL7R and the cell culture medium is supplemented with TSLP, they can survive without IL-3.

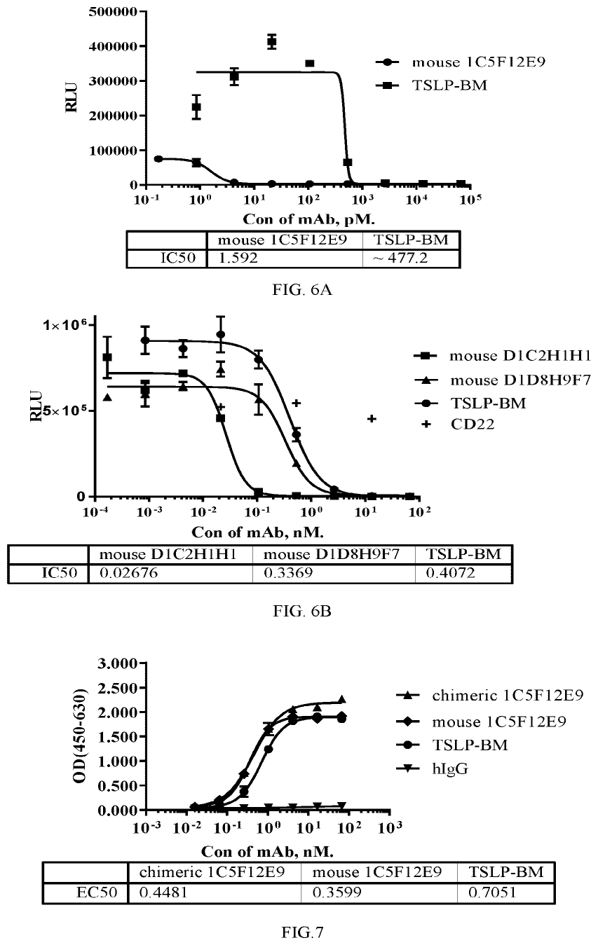

The anti-TSLP antibodies were further tested for their bioactivities of inhibiting proliferation of BAF3-3E6 cells expressing TSLPR (SEQ ID NO.: 33)/IL7R (SEQ ID NO.: 34) by using a cell-based functional assay. Briefly, 8×10³ BAF3-3E6 cells at the log phase stage in 100 μL RPMI1640 medium (Gibco®, Cat #A10491-01) supplemented with 10% FBS (Gibco®, Cat #10099-141) were plated onto 96-well plates. Then, 50 μL human TSLP-his proteins (SEQ ID NO.: 28, prepared in-house, 6.4 ng/mL μM in RPMI-1640) were mixed with 50 μL of the anti-TSLP antibodies or the control (starting from 40 μg/mL, 5-fold serial dilution in the culture medium), and the mixtures were incubated for 30 minutes at room temperature. Then, the antibody/TSLP-his mixtures (100 μL) were added to BAF3-3E6 cells plates, and incubated in a CO2 incubator at 37° C. for 72 hours. Thereafter, the cells plates were incubated with Cell Titer-Glo® Luminescent Cell Viability Assay Kit (Promega®, Cat #G7572, 50 μl/well) for 10 minutes at 37° C. Chemiluminescence was measured using a Tecan Infinite® 200 Pro® equipment. Data was analyzed using Graphpad Prism® software and IC₅₀ values were reported. The results of some antibodies were shown in FIGS. 6A-6B.

The data showed that all anti-TSLP antibodies were capable of blocking human TSLP binding to human TSLPR/IL7R, and the blocking activities were comparable to or a bit better than that of the benchmark. As shown in FIGS. 3A-3B, in the competitive ELISA, the IC₅₀ values of D1C2H1H1 and 1C5F12E9 were lower than that of the benchmark, indicating that they inhibited TSLP binding to TSLPR/IL7R more efficiently. In the cell based ligand blocking assay, as shown in FIGS. 5A-5B, the antibody D1D8H9F7 showed partial blocking on TSLP-TSLPR/IL7R binding, while antibodies D1C2H1H1 and 1C5F12E9 completely inhibited TSLP-TSLPR/IL7R binding with similar IC₅₀ as compared with the benchmark.

FIGS. 4A-4B showed that the anti-TSLP antibody D1D8H9F7 cannot block benchmark binding to human TSLP, indicating it might bind to a different epitope compared to the benchmark. The rest anti-TSLP antibodies that were capable of blocking benchmark-TSLP binding might bind to the same or similar epitope as the benchmark (Tezepelumab) did.

Further, all anti-TSLP antibodies were able to block TSLP-TSLPR/IL7R interaction, causing TSLP pathway blocking and BAF3-3E6 cell death, at a low antibody concentration, while the benchmark blocked TSLP-TSLPR/IL7R interaction at a high antibody level, as seen in FIGS. 6A-6B showing three representative antibodies' blocking curves.

### Example 5 Generation and Characterization of Exemplary Chimeric Antibody

The heavy and light chain variable regions of all the antibodies tested above were sequenced. Surprisingly, the antibodies other than D1D8H9F7 and D1C2H1H1 that were screened in a same cell fusion library were found to have the same heavy chain variable region and light chain variable region sequences. The inventors then rescreened the same cell fusion library, and antibodies that were picked out showing good properties were proved to have the same heavy/light chain variable regions. Without wishing to be bound to the theory, the inventors believe that the mAbs with such heavy/light chain variable regions were the dominate ones. The antibody 1C5F12E9 was picked as the representative for further characterization.

The heavy and light chain variable regions of the anti-TSLP mouse mAb 1C5F12E9 were cloned in frame to human IgGl heavy chain constant region (SEQ ID NO.: 12) and human kappa light chain constant region (SEQ ID NO.: 14), respectively, wherein the C-terminus of the variable region was linked to the N-terminus of the corresponding constant region. The vectors each containing a nucleotide encoding a heavy chain variable region linked to human IgGl heavy-chain constant region, and the vectors each containing a nucleotide encoding a light chain variable region linked to human kappa light-chain constant region were transiently transfected into 50 ml of 293F suspension cell cultures in a ratio of 1.1:1 light to heavy chain construct, with 1 mg/mL PEI. Cell supernatants were harvested after six days in shaking flasks, spun down to pellet cells, and then chimeric antibodies were purified from the cell supernatants. The purified chimeric antibodies were confirmed in the capture ELISA, Octet® affinity test, cell-based ligand-blocking FACS test and a cell-based functional assay following the protocols in the foregoing Examples and those described below.

The binding affinity of the purified chimeric 1C5F12E9 antibodies to human TSLP was assessed by Octet®. Briefly, APS biosensors (from ForteBio®) were presoaked with PBS for 1 hour, and then dipped in a well with PBS for 180 seconds. Then, the sensors were dipped in a well with human TSLP-his protein (SEQ ID NO.: 28, prepared in-house) at 5 μg/mL in running buffer for 760 seconds, and then dipped in a well with PBS for 600 seconds. Thereafter, the sensors were dipped in a well with HBS-EP⁺ buffer (provided by Biacore®) for 600 seconds. Finally, the sensors were dipped in a well with serially diluted mouse or chimeric 1C5F12E9 anti-TSLP antibodies (2-fold dilution in HBS-EP⁺ buffer, starting at 16.67 nM). The antigen-antibody association kinetics was followed for 2 minutes and the dissociation kinetics was followed for 3 minutes. The association and dissociation curves were fit to a 1:1 Langmuir binding model using ForteBio® Data Analysis 8.1 evaluation software. The Kₐ, K_{d} and K_{D} values were determined and summarized in Table 3 below. The binding affinity of the chimeric 1C5F12E9 antibodies was similar to that of the mouse antibody and the benchmark.

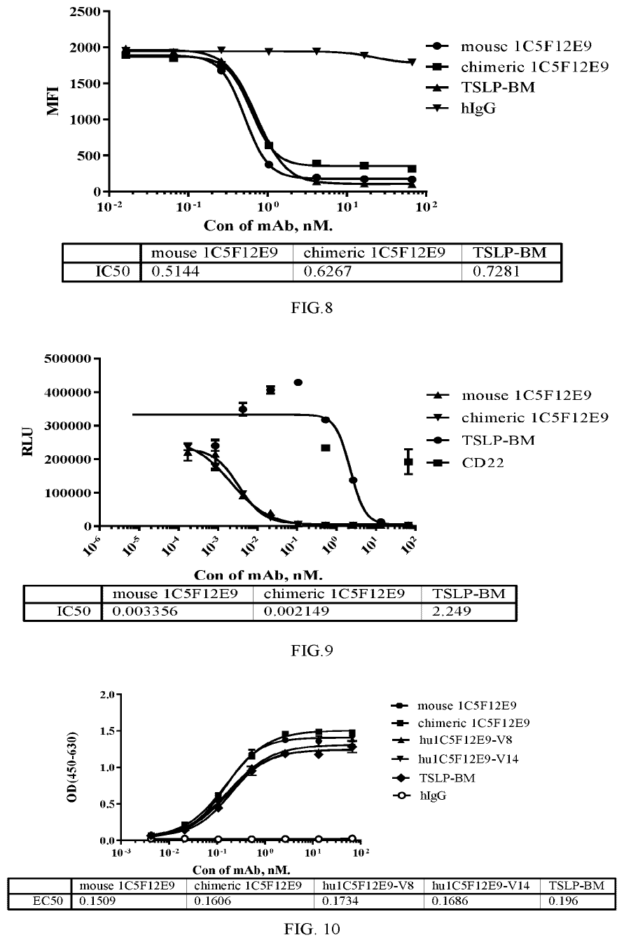

The results of the other three assays were shown in FIGS. 7 to 9. The chimeric 1C5F12E9 antibody showed similar TSLP binding capacity, blocking activity on TSLP-TSLPR/IL7R interaction and inhibitory effect on BAF3-3E6 cell survival to the mouse 1C5F12E9 antibody, but better than the benchmark.

TABLE 3
Binding Affinity of Chimeric Antibody

| | Kinetics on Octet ® | | |
|---|---|---|---|
| | Kₐ | K_{d} | K_{D} |
| mAb ID# | (M⁻¹s⁻¹) | (s⁻¹) | (M) |
| Mouse 1C5F12E9 | 6.00E+05 | 1.35E04 | 2.26E−10 |
| Chimeric 1C5F12E9 | 7.15E+05 | 1.59E−04 | 2.22E−10 |
| Tezepelumab | 6.60E+05 | 1.49E−04 | 2.26E−10 |

### Example 6 Humanization of Anti-TSLP Mouse Monoclonal Antibody

The mouse anti-TSLP 1C5F12E9 antibody was humanized. Humanization of the mouse antibody was conducted using the well-established CDR-grafting method as described in detail below.

To select acceptor frameworks for humanization of the mouse 1C5F12E9 antibody, the light and heavy chain variable region sequences of the antibody were blasted against the human immunoglobulin gene database. The human germline with the highest homology to the mouse antibody was selected as the acceptor frameworks for humanization. The mouse antibody heavy/light chain variable region CDRs were inserted into the selected frameworks and the residue(s) in the frameworks was/were further mutated to obtain more candidate heavy chain/light chain variable regions. A total of 18 exemplary humanized 1C5F12E9 antibodies, namely from hulC5F12E9-V1 to hulC5F12E9-V18, were obtained whose heavy/light chain variable region sequences were in Table 1.

The vectors containing nucleotide sequences encoding humanized 1C5F12E9 heavy chain chain variable region plus human IgG4 heavy-chain ((SEQ ID NO: 13) and humanized 1C5F12E9 light chain chain variable region plus human kappa light-chain constant regions (SEQ ID NO: 14) were transiently transfected into 50 ml of 293F suspension cell cultures in a ratio of 60% to 40% light to heavy chain construct, with 1 mg/ml PEI Cell supernatants containing humanized antibodies were harvested after six days in shaking flasks, spun down to pellet cells, and then 18 antibodies were purified from the cell supernatants.

### Example 7 Characterization of Exemplary Humanized Anti-TSLP Monoclonal Antibodies

The binding affinities and binding kinetics of the exemplary purified humanized 1C5F12E9 antibodies to human TSLP were assessed by Biacore® T200 system (GE Healthcare™ Pittsburgh, PA, USA) following the protocol in Example 2.

TABLE 4
Binding Affinities of Humanized 1C5F12E9 mAbs

| | Kinetics on Biacore ® | | |
|---|---|---|---|
| | Human TSLP | | |
| | Kₐ | K_{d} | K_{D} |
| mAb | (M⁻¹s⁻¹) | (s⁻¹) | (M) |
| hu1C5F12E9-V1 | 9.93E+04 | 9.07E−08 (<1.00E−05) | <1.01E−10 |
| hu1C5F12E9-V3 | 1.03E+05 | 1.94E−08 (<1.00E−05) | <9.71E−11 |
| hu1C5F12E9-V5 | 1.03E+05 | 7.89E−08 (<1.00E−05) | <9.71E−11 |
| hu1C5F12E9-V7 | 1.08E+05 | 3.16E−08 (<1.00E−05) | <9.26E−11 |
| hu1C5F12E9-V9 | 1.22E+05 | 6.57E−07 (<1.00E−05) | <8.20E−11 |
| hu1C5F12E9-V11 | 9.07E+04 | 7.56E−07 (<1.00E−05) | <1.10E−10 |
| hu1C5F12E9-V12 | 9.61E+04 | 5.30E−08 (<1.00E−05) | <1.04E−10 |
| hu1C5F12E9-V13 | 1.04E+05 | 3.62E−07 (<1.00E−05) | <9.62E−11 |
| hu1C5F12E9-V14 | 1.05E+05 | 3.21E−06 (<1.00E−05) | <9.56E−11 |
| hu1C5F12E9-V15 | 1.12E+05 | 3.63E−06 (<1.00E−05) | <8.93E−11 |
| hu1C5F12E9-V16 | 1.04E+05 | 1.17E−06 (<1.00E−05) | <9.62E−11 |
| hu1C5F12E9-V17 | 1.10E+05 | 6.43E−06 (<1.00E−05) | <9.09E−11 |
| hu1C5F12E9-V18 | 1.21E+05 | 9.65E−06 (<1.00E−05) | <8.26E−11 |
| Chimeric 1C5F12E9 | 6.96E+04 | 6.36E−05 | 9.14E−10 |
| Tezepelumab | 1.99E+05 | 1.62E−04 | 8.15E−10 |

The Kₐ, K_{d} and K_{D} values were determined and summarized in Table 4 above.

The lower limit of K_{d} as measured by Biacore® is 1.00E-05, and a K_{d} below 1.00E-05 can be roughly calculated from the corresponding sensorgram. The results indicated that all humanized 1C5F12E9 antibodies had higher binding affinities to human TSLP them Tezepelumab.

### Example 8 Characterization of Humanized Anti-TSLP Antibodies huC5F12E9-V8 and huC5F12E9-V14

The humanized antibodies hulC5F12E9-V8 and huC5F12E9-V14 were selected for further characterization. Specifically, they were tested for the binding affinities/capacities to human and cynomolgus TSLPs and other functions by Biacore, capture ELISA, indirect ELISA, competitive ELISAs, cell-based ligand blocking FACS and functional assays, following the protocols described in Examples 2 to 4, and the results were shown in Table 5 below and FIGS. 10-14, and 15A-15B.

The data showed that hulC5F12E9-V8 and hulC5F12E9-V14 showed comparable in vitro activities to the parent mouse and chimeric antibodies.

It can be seen from Table 5, FIG. 10 and FIG. 11 that the humanized antibodies hulC5F12E9-V8 and hulC5F12E9-V14 showed higher binding affinity/activity to human TSLP, and comparable binding affinity/capacity to cynomolgus TSLP when compared to the benchmark.

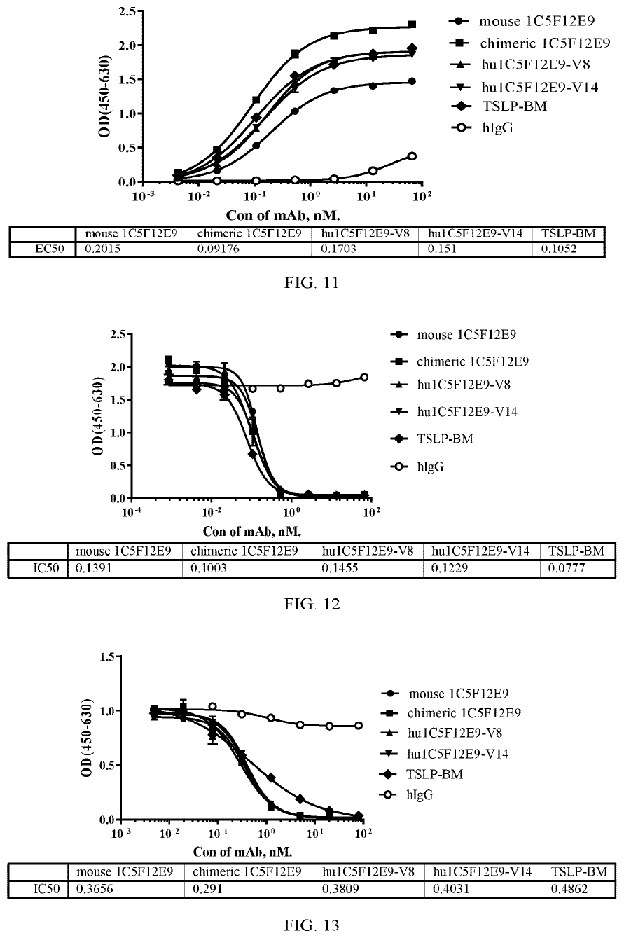

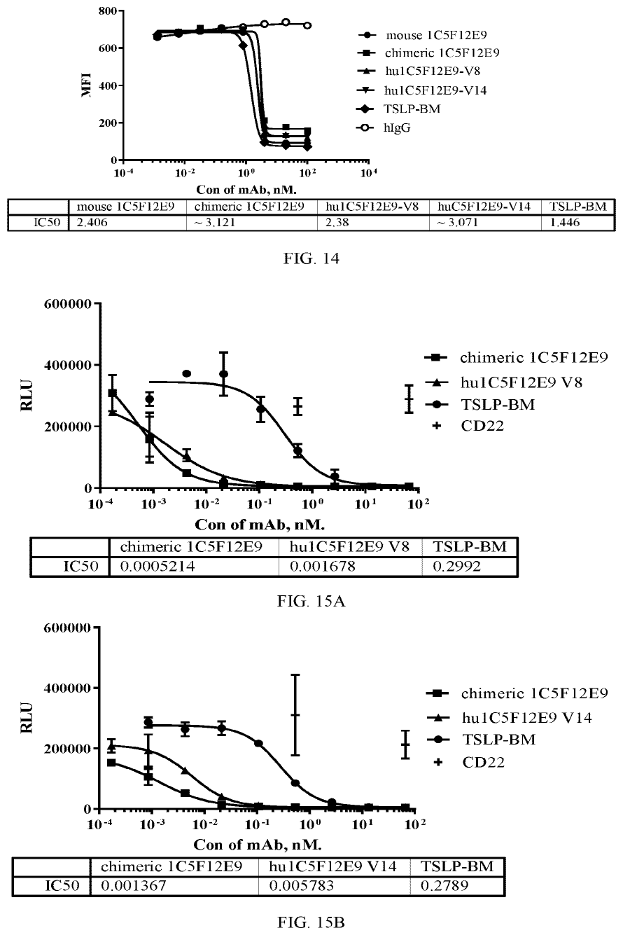

FIGS. 12 and 14 showed that the humanized antibodies hulC5F12E9-V8 and hulC5F12E9-V14 were capable of blocking human TSLP binding to human TSLPR/IL7R.

FIGS. 15A-15B showed that the humanized antibodies hulC5F12E9-V8 and hulC5F12E9-V14 were able to block TSLP-TSLPR/IL7R interaction, causing TSLP pathway blocking and death of BAF3-3E6 cells at a low antibody concentration, while the benchmark showed such a capacity at a high antibody level.

TABLE 5
Binding Affinites of hu1C5F12E9-V8 and hu1C5F12E9-V14 mAbs

| | Kinetics on BIAcore ® | | | | | |
|---|---|---|---|---|---|---|
| | Human TSLP | | | Cynomolgus TSLP | | |
| | Kₐ | K_{d} | K_{D} | Kₐ | K_{d} | K_{D} |
| mAbs | (1/Ms) | (s − 1) | (M) | (1/Ms) | (s − 1) | (M) |
| mouse 1C5F12E9 | 1.04E+05 | <1.00E−05 | <9.66E−11 | 2.89E+05 | <1.00E−05 | <3.46E−11 |
| chimeric 1C5F12E9 | 1.93E+05 | <1.00E−05 | <5.17E−11 | 3.83E+05 | <1.00E−05 | <2.61E−11 |
| hu1C5F12E9-V8 | 1.67E+05 | <1.00E−05 | <5.98E−11 | 3.05E+05 | <1.00E−05 | <3.28E−11 |
| hu1C5F12E9-V14 | 1.64E+05 | <1.00E−05 | <6.10E−11 | 3.11E+05 | <1.00E−05 | <3.22E−11 |
| Tezepelumab | 4.24E+05 | 9.84E−05 | 2.32E−10 | 8.49E+05 | 5.71E−05 | 6.72E−11 |

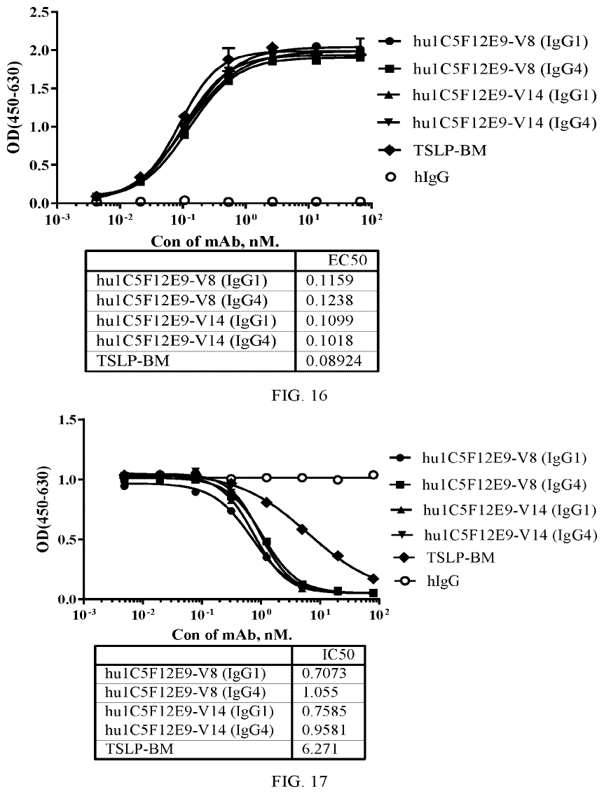

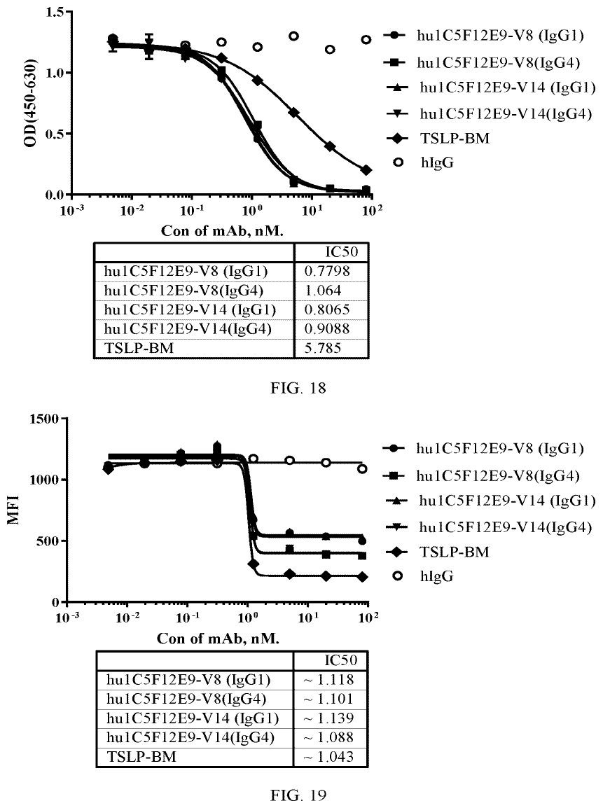

Thereafter, the humanized antibodies hulC5F12E9-V8 and hulC5F12E9-V14 having human IgGl heavy chain constant region (SEQ ID NO.: 12) and human kappa constant region (SEQ ID NO.: 14) were compared with hulC5F12E9-V8 and hulC5F12E9-V14 having human IgG4 heavy chain constant region (SEQ ID NO.: 13) and human kappa constant region (SEQ ID NO.: 14) in binding affinity/capacity to human and cynomolgus TSLPs and other functions by Biacore, capture ELISA, competition ELISA, cell based ligand blocking FACS, cell based functional assay, cell based reporter assay and protein thermal shift assay, following the protocols described in Examples 2 to 4 and those described below. The results were shown in FIGS. 16-19, 20A-20B, 21A-21B and 22.

To determine the thermal stabilities of the four anti-TSLP humanized antibodies, a protein thermal shift assay was used to determine Tm (melting temperature) using a GloMelt™ Thermal Shift Protein Stability Kit (Biotium®, Cat #33022-T). Briefly, the GloMelt™ dye was allowed to thaw and reach room temperature. The vial containing the dye was vortexed and centrifuged. Then, 10× dye was prepared by adding 5 μL 200× dye to 95 μL PBS. 2 μL 10× dye and 10 μg humanized antibodies were added, and PBS was added to a total reaction volume of 20 μL. The tubes containing the dye and antibodies were briefly spun and placed in real-time PCR thermocycler (Roche®, LightCycler® 480 II) set up with a melt curve program having the parameters in Table 6.

TABLE 6
Parameters for Melt Curve Program

| | | Ramp | Holding |
|---|---|---|---|
| Profile step | Temperature | rate | Time |
| Initial hold | 25° C. | NA | 30 s |
| Melt curve | 25-99° C. | 0.1° C./s | NA |

In the cell based reporter assay, a reporter cell line HEK293T-TSLPR/IL7R/STAT5-Luc was used that expressed cell-surface human TSLPR (SEQ ID NO.: 33) and human IL7R (SEQ ID NO.: 34). The HEK293T-TSLPR/IL7R/STAT5-Luc cells were prepared in house, following the instruction of lipofectamine 3000 transfection reagent (Thermo Fisher®), by transfecting HEK293T cells (ATCC® CRL-11268) with pCMV-T-P plasmids inserted with TSLPR coding sequence between EcoRI and Xbal sites, pCMV3-SP plasmids inserted with IL7R coding sequence between HindIII and Xbal sites and pGL4.52 [luc2P/STAT5RE/Hygro](Promega®).

Briefly, HEK293T-TSLPR/IL7R/STAT5-Luc cells were harvested from cell culture flasks. Then, 5×10⁴ cells in 100 μL DMEM medium (Gibco®, Cat #10566-016) supplemented with 10% FBS (Gibco®, Cat #10099-141) were plated onto the 96 well cell culture plates (Corning®, Cat #30218026). Meanwhile, 50 μL human TSLP-his (SEQ ID NO.: 28, 160 ng/mL in DMEM medium supplemented with 10% FBS) was respectively mixed with 50 μL serially diluted anti-TSLP antibodies hulC5F12E9-V8(IgGl), hulC5F12E9-V8(IgG4), hulC5F12E9-V14(IgGl), hulC5F12E9-V14(IgG4), and Tezepelumab (5-fold dilution in DMEM medium supplemented with 10% FBS, starting at 200 μg/mL), and then incubated for 30 minutes at room temperature. Then, the anti-TSLP antibody/TSLP-his mixtures were added to the plates, 100 μl/well, and the plates were incubated in a CO2 incubator at 37° C. for 16-18 hours. The supernatants were discarded 100 μl/well, and then Luciferase detection Reagent (50 μL/well, Promega®, Cat #E6120) was added. Ten minutes later, the plates were subject to analysis by Tecan Infinite 200Pro® plate-reader. Data of luminescence signal were analyzed using Graphpad Prism® software and IC₅₀ values were reported.

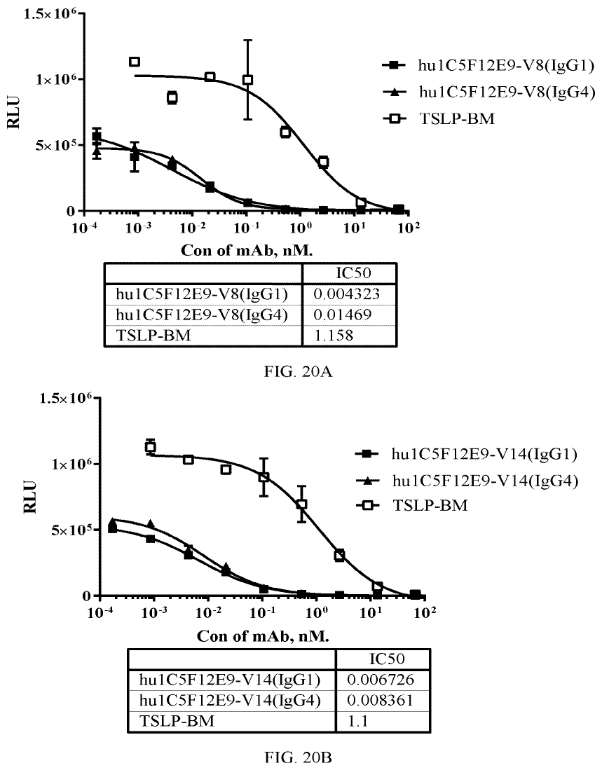

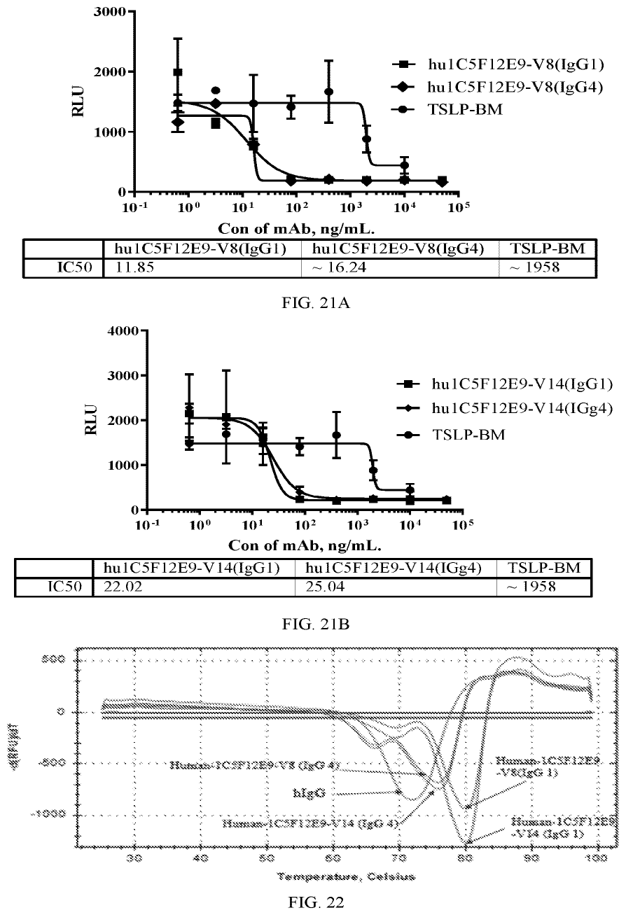

As showed in FIGS. 16, 17, 19, 20A-20B, and 21A-21B, hulC5F12E9-V8 (IgGl), hulC5F12E9-V8 (IgG4), hulC5F12E9-V14 (IgGl) and hulC5F12E9-V14 (IgG4) showed comparable or a bit better in vitro activities as compared to Tezepelumab. Especially, the data in FIGS. 20A-20B and 21A-21B showed the humanized antibodies were able to block TSLP-TSLPR/IL7R interaction, causing TSLP pathway blocking and death of BAF3-3E6 cells, at a much lower antibody concentration as compared to the benchmark.

As shown in FIG. 22, the melting temperatures (T1 and T2) of hulC5F12E9-V8 (IgGl), hulC5F12E9-V8 (IgG4), hulC5F12E9-V14 (IgGl) and hulC5F12E9-V14 (IgG4) were (69.5° C., 80° C.), (66.5° C., 76° C.), (69.5° C., 80° C.), (66.5° C., 76° C.), respectively.

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/Sequence/SEQ ID NO.
VH-CDR1 for mouse, chimeric and humanized 1C5F12E9 antibodies
(SEQ ID NO: 1)
TYWMH
VH-CDR2 for mouse, chimeric and humanized 1C5F12E9 antibodies
(SEQ ID NO: 2)
VIDPSDSDTTYNQKFKG
VH-CDR3 for mouse, chimeric and humanized 1C5F12E9 antibodies
(SEQ ID NO: 3)
SLDGYYDY
VL-CDR1 for mouse, chimeric and humanized IC5F12E9 antibodies
(SEQ ID NO: 4)
RPTENIYSYLA
VL-CDR2 for mouse, chimeric and humanized 1C5F12E9 antibodies
(SEQ ID NO: 5)
FARTLAE
VL-CDR3 for mouse, chimeric and humanized 1C5F12E9 antibodies
(SEQ ID NO: 6)
QHHYGTPWT
VH for mouse and chimeric IC5F12E9 antibodies
(SEQ ID NO: 7)
QVQLQQPGTELVKPGASVKMSCKASGYTFTTYWMHWVKQRPGQGLEWIGVIDPSDSDTTYNQK
FKGKATLTVDTSSSTAYMQLSSLTSEDSAVYYCTRSLDGYYDYWGQGTTLTVSS
(SEQ ID NO: 17)
CAGGTCCAACTGCAGCAGCCTGGGACTGAGCTGGTGAAGCCTGGGGCTTCAGTGAAGATGTC
CTGCAAGGCTTCTGGCTACACCTTCACCACCTACTGGATGCACTGGGTGAAGCAGAGGCCTG
GACAAGGCCTTGAGTGGATCGGAGTGATTGATCCTTCTGATAGTGATACTACCTACAATCAAA
AGTTCAAGGGCAAGGCCACATTGACTGTAGACACATCCTCCAGCACAGCCTACATGCAGCTC
AGCAGCCTGACATCTGAGGACTCTGCGGTCTATTACTGTACAAGGTCCCTTGATGGTTACTAC
GACTACTGGGGCCAAGGCACCACTCTCACAGTCTCCTCA
(SEQ ID NO: 18)
CAGGTGCAGCTGCAGCAGCCCGGCACCGAGCTGGTGAAGCCTGGCGCTAGCGTGAAGATGTC
CTGTAAGGCCAGCGGCTACACATTCACTACATACTGGATGCACTGGGTGAAGCAGAGACCTG
GCCAGGGCCTGGAGTGGATCGGCGTGATCGACCCCAGCGACTCCGATACCACCTACAACCAG
AAGTTTAAGGGCAAGGGCACCCTGACAGTGGATACAAGCTCCTCCACAGCCTACATGCAGCT
GTCCAGCCTGACCTCCGAGGATTCCGCCGTGTACTACTGCACAAGGTCCCTGGATGGCTACTA
CGATTACTGGGGCCAGGGCACAACCCTGACAGTCAGCAGC
VH for hu1C5F12E9-V1, hu1C5F12E9-V7, and hu1C5F12E9-V13
(SEQ ID NO: 20)
GATTTATTACTGTCAACACCATTATGGTACTCCGTGGACGTTCGGTGGAGGCACCAAGCTGGA
AATCAAA
(SEQ ID NO: 21)
GACATCCAGATGACCCAGTCCCCCGCCAGCCTGTCCGCCTCTGTGGGAGAGACCGTGACCAT
CACATGCAGGCCCACCGAGAATATCTACTCCTACCTGGCCTGGTATCAACAGAAGCAGGGCA
AGAGCCCTCACCTGCTGGTGTACTTCGCCAGGACACTGGCCGAGGGCGTGCCCTCTAGGTTCA
GCGGCAGCGGCTCCGGCACACAGTTTTCCCTGAAGATCAACAGCCTGCAGCCTGAGGATTTT
GGCATCTACTACTGCCAGCACCACTACGGCACACCCTGGACCTTTGGCGGCGGCACCAAGCT
GGAGATCAAG
VL for hu1C5F12E9-V1 - hu1C5F12E9-V6
(SEQ ID NO: 11, X1 = S, X2 = V)
DIQMTQSPSSLSASVGDRVTITCRPTENIYSYLAWYQQKPGKX1PKLLX2YFARTLAEGVPSRFSGS
GSGTDFTLTISSLQPEDFATYYCQHHYGTPWTFGGGTKVEIK
DIQMTQSPSSLSASVGDRVTITCRPTENIYSYLAWYQQKPGKSPKLLVYFARTLAEGVPSRFSGSG
SGTDFTLTISSLQPEDFATYYCQHHYGTPWTFGGGTKVEIK
VL for hu1C5F12E9-V7 - hu1C5F12E9-V12
(SEQ ID NO: 11, X1 = A, X2 = I)
DIQMTQSPSSLSASVGDRVTITCRPTENIYSYLAWYQQKPGKX1PKLLX2YFARTLAEGVPSRFSGS
GSGTDFTLTISSLQPEDFATYYCQHHYGTPWTFGGGTKVEIK
DIQMTQSPSSLSASVGDRVTITCRPTENIYSYLAWYQQKPGKAPKLLIYFARTLAEGVPSRFSGSGS
GTDFTLTISSLQPEDFATYYCQHHYGTPWTFGGGTKVEIK
(SEQ ID NO: 22)
GATATCCAGATGACACAGAGCCCCAGCAGCCTGAGCGCCAGCGTGGGAGACAGGGTGACAA
TCACATGCAGACCCACCGAGAATATCTACAGCTACCTGGCCTGGTATCAACAGAAGCCTGGC
AAGGCCCCCAAGCTGCTGATCTACTTCGCCAGAACCCTGGCCGAGGGCGTGCCCTCTAGGTTC
AGCGGCTCCGGCAGCGGCACCGACTTCACACTGACCATCTCCTCCCTGCAGCCCGAGGACTTC
GCCACATACTACTGCCAGCACCACTACGGCACACCTTGGACCTTCGGCGGCGGCACAAAGGT
GGAGATCAAG
VL for hu1C5F12E9-V13 - hu1C5F12E9-V18
(SEQ ID NO: 11, X1 = S, X2 = I)
DIQMTQSPSSLSASVGDRVTITCRPTENIYSYLAWYQQKPGKX1PKLLX2YFARTLAEGVPSRFSGS
GSGTDFTLTISSLQPEDFATYYCQHHYGTPWTFGGGTKVEIK
DIQMTQSPSSLSASVGDRVTITCRPTENIYSYLAWYQQKPGKSPKLLIYFARTLAEGYPSRFSGSGS
GTDFTLTISSLQPEDFATYYCQHHYGTPWTFGGGTKVEIK
Human IgG1 heavy chain constant region
(SEQ ID NO: 12)
ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLS
LSPGK
(SEQ ID NO: 23)
GCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAA
CTCAGGCGCCCTGACCAGCGGCGTGCACACCTTCCCGGCTGTCCTACAGTCCTCAGGACTCTA
CTCCCTCAGCAGCGTGGTGACCGTGCCCTCCAGCAGCTTGGGCACCCAGACCTACATCTGCAA
CGTGAATCACAAGCCCAGCAACACCAAGGTGGACAAGAAAGTTGAGCCCAAATCTTGTGACA
AAACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAAC
CACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAATGA
(SEQ ID NO: 57)
ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSYMHEALHNHYTQKSLS
LSPQK
(SEQ ID NO: 58)
GCCTCCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAA
CTCAGGCGCCCTGACCAGCGGCGTGCACACCTTCCCGGCTGTCCTACAGTCCTCAGGACTCTA
CTCCCTCAGCAGCGTGGTGACCGTGCCCTCCAGCAGCTTGGGCACCCAGACCTACATCTGCAA
CGTGAATCACAAGCCCAGCAACACCAAGGTGGACAAGAAAGTTGAGCCCAAATCTTGTGACA
AAACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGGGTGGTCAGC
GTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACAG
CAAGCTCACCGTGGACAAGACCAGGTGGCAGCAGGGGAACCTCTTCTCATGCTCCGTGATGC
ATGAGGCTCTGCACAACCACTACACGCAGAAGAGCCTCTCCCTGTCTCCGGGTAAATGA
Human IgG4 heavy chain constant region
(SEQ ID NO: 13)
ASTKGPSVFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLGK
(SEQ ID NO: 24)
GCCAGCACAAAGGGCCCTTCCGTGTTTCCCCTGGCCCCCTGCAGCAGGAGCACCTCTGAGTCC
ACCGCCGCCCTGGGCTGTCTGGTGAAGGACTACTTTCCCGAGCCCGTGACCGTGAGCTGGAAT
TCCGGCGCCCTGACATCCGGCGTGCACACCTTCCCCGCCGTGCTGCAGTCCTCCGGCCTGTAC
AGCCTGAGCTCCGTGGTGACAGTGCCTTCCTCCTCCCTGGGCACCAAGACCTACACATGTAAT
GTGGATCACAAGCCCAGCAACACAAAGGTGGATAAGAGAGTGGAGTCCAAGTACGGCCCTC
CTTGCCCTCCCTGTCCTGCCCCAGAGTTCCTGGGCGGCCCCTCTGTGTTCCTGTTCCCCCCTAA
GCCCAAGGACACACTGATGATCTCCAGGACCCCTGAGGTGACCTGCGTGGTGGTGGACGTGA
GCCAGGAGGACCCTGAGGTGCAGTTCAATTGGTACGTGGATGGCGTGGAGGTGCACAATGCC
AAGACAAAGCCCAGAGAGGAGCAGTTTAATTCCACATACAGGGTGGTGTCCGTGCTGACCGT
GCTGCACCAGGATTGGCTGAACGGCAAGGAGTACAAGTGTAAGGTGAGCAACAAGGGCCTG
CCTTCCTCCATCGAGAAGACAATCAGCAAGGCCAAGGGCCAGCCTAGGGAGCCCCAGGTGTA
CACACTGCCTGCCAGCCAGGAGGAGATGACCAAGAACCAGGTGAGCCTGACCTGCCTGGTGA
AGGGCTTCTACCCTAGCGACATCGCCGTGGAGTGGGAGTCCAACGGCCAGCCCGAGAATAAC
TACAAGACAACACCCCCCGTGCTGGATTCCGATGGCAGCTTCTTTCTGTACTCCAGGCTGACC
GTGGATAAGAGCAGGTGGCAGGAGGGCAATGTGTTCAGCTGCTCCGTGATGCACGAGGCCCT
GCACAATCACTACACCCAGAAGAGCCTGTCCCTGAGCCTGGGCAAGTGA
Human kappa light chain constant region
(SEQ ID NO: 14)
RTVAAPSVFIFPPSDEQLKSGTASVVCLLNNFYPREAKVQWKYDNALQSGNSQESVTEQDSKDST
YSLSSTLTLSKADYEKHKVYACEVTHQGLSSPVTKSFNRGEC
(SEQ ID NO: 25)
CGTACGGTGGCGGCGCCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAA
AGTCTACGCCTGCGAAGTCACCCATCAGGGCCTGAGCTCGCCCGTCACAAAGAGCTTCAACA
GGGGAGAGTGTTGA
Mouse IgG1 heavy chain constant region
(SEQ ID NO: 15)
AKTTPPSVYPLAPGSAAQTNSMVTLGCLVKGYFPEPVTVTWNSGSLSSGVHTFPAVLQSDLYTLS
SSVTVPSSTWPSETVTCNVAHPASSTKVDKKIVPRDCGCKPCICTVPEVSSVFIFPPKPKDVLTITLT
PKVTCVVVDISKDDPEVQFSWFVDDVEVHTAQTQPREEQFNSTFRSVSELPIMHQDWLNGKEFKC
RVNSAAFPAPIEKTISKTKGRPKAPQVYTIPPPKEQMAKDKVSLTCMITDFFPEDITVEWQWNGQP
AENYKNTQPIMDTDGSYFVYSKLNVQKSNWEAGNTFTCSVLHEGLHNHHTEKSLSHSPGK
(SEQ ID NO: 26)
GCCAAAACGACACCCCCATCTGTCTATCCACTGGCCCCTGGATCTGCTGCCCAAACTAACTCC
ATGGTGACCCTGGGATGCCTGGTCAAGGGCTATTTCCCTGAGCCAGTGACAGTGACCTGGAA
CTCTGGATCCCTGTCCAGCGGTGTGCACACCTTCCCAGCTGTCCTGCAGTCTGACCTCTACACT
CTGAGCAGCTCAGTGACTGTCCCCTCCAGCACCTGGCCCAGCGAGACCGTCACCTGCAACGTT
GCCCACCCGGCCAGCAGCACCAAGGTGGACAAGAAAATTGTGCCCAGGGATTGTGGTTGTAA
GCCTTGCATATGTACAGTCCCAGAAGTATCATCTGTCTTCATCTTCCCCCCAAAGCCCAAGGA
TGTGCTCACCATTACTCTGACTCCTAAGGTCACGTGTGTTGTGGTAGACATCAGCAAGGATGA
TCCCGAGGTCCAGTTCAGCTGGTTTGTAGATGATGTGGAGGTGCACACAGCTCAGACGCAAC
CCCGGGAGGAGCAGTTCAACAGCACTTTCCGCTCAGTCAGTGAACTTCCCATCATGCACCAG
GACTGGCTCAATGGCAAGGAGTTCAAATGCAGGGTCAACAGTGCAGCTTTCCCTGCCCCCAT
CGAGAAAACCATCTCCAAAACCAAAGGCAGACCGAAGGCTCCACAGGTGTACACCATTCCAC
CTCCCAAGGAGCAGATGGCCAAGGATAAAGTCAGTCTGACCTGCATGATAACAGACTTCTTC
CCTGAAGACATTACTGTGGAGTGGCAGTGGAATGGGCAGCCAGCGGAGAACTACAAGAACA
CTCAGCCCATCATGGACACAGATGGCTCTTACTTCGTCTACAGCAAGCTCAATGTGCAGAAGA
GCAACTGGGAGGCAGGAAATACTTTCACCTGCTCTGTGTTACATGAGGGCCTGCACAACCAC
CATACTGAGAAGAGCCTCTCCCACTCTCCTGGTAAATGA
Mouse kappa light chain constant region
(SEQ ID NO: 16)
RADAAPTVSIFPPSSEQLTSGGASVVCFLNNFYPKDINVKWKIDGSERQNGVLNSWTDQDSKDST
YSMSSTLTLTKDEYERHNSYTCEATHKTSTSPIVKSFNRGEC
(SEQ ID NO: 27)
CGGGCTGATGCTGCACCAACTGTATCCATCTTCCCACCATCCAGTGAGCAGTTAACATCTGGA
GGTGCCTCAGTCGTGTGCTTCTTGAACAACTICTACCCCAAAGACATCAATGTCAAGTGGAAG
ATTGATGGCAGTGAACGACAAAATGGCGTCCTGAACAGTTGGACTGATCAGGACAGCAAAGA
CAGCACCTACAGCATGAGCAGCACCCTCACGTTGACTAAGGACGAGTATGAACGACATAACA
GCTATACCTGTGAGGCCACTCACAAGACATCAACTTCACCCATTGTCAAGAGCTTCAACAGG
GGAGAGTGT
Recombinant human TSLP-his
(SEQ ID NO: 28)
YDFTNCDFEKIKAAYLSTISKDLITYMSGTKSTEFNNTVSCSNRPHCLTEIQSLTFNPTAGCASLAK
EMFAMKTKAALAIWCPGYSETQINATQAMKKRRKRKVTTNKCLEQVSQLQGLWRRFNRPLLKQ
QHHHHHHHHHH
Recombinant cynomolgus monkey TSLP-his
(SEQ ID NO: 29)
MYDFTNCDFQKIEADYLRTISKDLITYMSGTKSTDFNNTVSCSNRPHCLTEIQSLTFNPTPRCASLA
KEMFARKTKATLALWCPGYSETQINATQAMKKRRKRKVTTNKCLEQVSQLLGLWRRFIRTLLKK
QLEHHHHHH
human TSLPR-Fc
(SEQ ID NO: 30)
GAAEGVQIQIIYFNLETVQVTWNASKYSRTNLTFHYRFNGDEAYDQCTNYLLQEGHTSGCLLDAE
QRDDILYFSIRNGTHPVFTASRWMVYYLKPSSPKHVRFSWHQDAVTVTCSDLSYGDLLYEVQYR
SPFDTEWQSKQENTCNVTIEGLDAEKCYSFWVRVKAMEDVYGPDTYPSDWSEVTCWQRGEIRD
ACAETPTPPKPKLSKDIEGRMDEPKSCDKTHTCPPCPAPELLGGPSVFLFPPKPKDTLMISRTPEVT
CVVVDVSHEDPEVKFNWYVDQVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
human IL7Ra-Fc
(SEQ ID NO: 31)
ESGYAQNGDLEDAELDDYSFSCYSQLEVNGSQHSLTCAFEDPDVNTTNLEFEICGALVEVKCLNF
RKLQEIYFIETKKFLLIGKSNICVKYGEKSLTCKKIDLTTIVKPEAPFDLSVIYREGANDFVVTFNTS
HLQKKYVKVLMHDVAYRQEKDENKWTHVNLSSTKLTLLQRKLQPAAMYEIKVRSIPDHYFKGF
WSEWSPSYYFRTPEINNSSGEPKSCDKTHTCPPCPAPELLGGPSVFLFPPKPKDTLMISRTPEVTCV
VVDVSHEDPEVKFNWYVDQVEVHNAKTKPREEQYNSTYRVVSVLTVLHQDWLNQKEYKCKVS
NKALPAPIEKTISKAKGQPREPQVYTLPPSREEMTKNQVSLTCLVKGFYPSDIAVEWESNGQPENN
YKTTPPVLDSDQSFFLYSKLTVDKSRWQQGNVFSCSVMHEALHNHYTQKSLSLSPGK
human TSLP-Fc
(SEQ ID NO: 32)
YDFTNCDFEKIKAAYLSTISKDLITYMSGTKSTEFNNTVSCSNRPHCLTEIQSLTFNPTAGCASLAK
EMFAMKTKAALAIWCPGYSETQINATQAMKKRRKRKVTTNKCLEQVSQLQGLWRRFNRPLLKQ
QEPKSCDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
human TSLPR
(SEQ ID NO: 33)
MGRLVLLWGAAVFLLGGWMALGQGGAAEGVQIQIIYFNLETVQVTWNASKYSRTNLTFHYRFN
GDEAYDQCTNYLLQEGHTSGCLLDAEQRDDILYFSIRNGTHPVFTASRWMVYYLKPSSPKHVRFS
WHQDAVTVTCSDLSYGDLLYEVQYRSPFDTEWQSKQENTCNVTIEGLDAEKCYSFWVRVKAME
DVYGPDTYPSDWSEVTCWQRGEIRDACAETPTPPKPKLSKFILISSLAILLMVSLLLLSLWKLWRV
KKFLIPSVPDPKSIFPGLFEIHQGNFQEWITDTQNVAHLHKMAGAEQESGPEEPLVVQLAKTEAESP
RMLDPQTEEKEASGGSLQLPHQPLQGGDVVTIGGFTFVMNDRSYVAL
Human IL7R
(SEQ ID NO: 34)
MTILGTTFGMVFSLLQVVSGESGYAQNGDLEDAELDDYSFSCYSQLEVNGSQHSLTCAFEDPDVN
TTNLEFEICGALVEVKCLNFRKLQEIYFIETKKFLLIGKSNICVKVGEKSLTCKKIDLTTIVKPEAPF
DLSVIYREGANDFVVTFNTSHLQKKYVKVLMHDVAYRQEKDENKWTHVNLSSTKLTLLQRKLQ
PAAMYEIKVRSIPDHYFKGFWSEWSPSYYFRTPEINNSSGEMDPILLTISILSFFSVALLVILACVLW
KKRIKPIVWPSLPDHKKTLEHLCKKPRKNLNVSFNPESFLDCQIHRVDDIQARDEVEGFLQDTFPQ
QLEESEKQRLGGDVQSPNCPSEDVVVTPESFGRDSSLTCLAGNVSACDAPILSSSRSLDCRESGKN
GPHVYQDLLLSLGTTNSTLPPPFSLQSGILTLNPVAQGQPILTSLGSNQEEAYVTMSSFYQNQ
Heavy chain of Tezepelumab
(SEQ ID NO.: 35)
QMQLVESGGGVVQPGRSLRLSCAASGFTFRTYGMHWVRQAPGKGLEWVAVIWYDGSNKHYAD
SVKQRFTITRDNSKNTLNLQMNSLRAEDTAVYYCARAPQWELVHEAFDIWGQGTMVTVSSASTK
GPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTP
EVTCVVVDVSHEDPEVQFNWYVDGVEVHNAKIKPREEQFNSTFRVVSVLTVVHQDWLNGKEY
KCKVSNKGLPAPIEKTISKTKGQPREPQVYTLPPSREEMTKNQVSLTCLVKGFYPSDIAVEWESNG
QPENNYKTTPPMLDSDGSFFLYSKLTVDKSRWQQQGNVFSCSVMHEALHNHYTQKSLSLSPGK
Light chain of Tezepelumab
(SEQ ID NO.: 36)
SYVLTQPPSYSVAPGQTARITCGGNNLGSKSVHWYQQKPGQAPVLVVYDDSDRPSWIPERFSGSN
SGNTAILTISRGEAGDEADYYCQVWDSSSDHVVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSC
QVTHEGSTVEKTVAPTECS
VH-CDR1 for mouse antibody D1D8H9F7
(SEQ ID NO.: 37)
SYYMN
VH-CDR2 for mouse antibody D1D8H9F7
(SEQ ID NO.: 38)
QIYPGDGETNYNGKFKD
VH-CDR3 for mouse antibody D1D8H9F7
(SEQ ID NO.: 39)
AGYSDYVYAMDY
VL-CDR1 for mouse antibody D1D8H9F7
(SEQ ID NO.: 40)
SVSSSITSSNLH
VL-CDR2 for mouse antibody D1D8H9F7
(SEQ ID NO.: 41)
GTSNLAS
VL-CDR3 for mouse antibody D1D8H9F7
(SEQ ID NO.: 42)
QQWSSFPL
VH for mouse antibody D1D8H9F7
(SEQ ID NO.: 43)
QVQLQQSGADLVRPGSSVKISCKTSGFAFSSYYMNWVKQRPGQGLEWIGQIYPQDGETNYNGKF
KDKATLTADKSSSTVYMQLSSLTSEDSAVYFCARAGYSDYVYAMDYWGQGTSYTVSS
(SEQ ID NO.: 45)
CAGGTTCAGCTGCAGCAGTCTGGGGCTGACCTGGTGAGGCCTGGGTCCTCAGTGAAGATTTCC
TGCAAGACTTCTGGCTTTGCATTCAGTAGCTACTATATGAACTGGGTGAAGCAGAGGCCTGGA
CAGGGTCTTGAGTGGATTGGACAGATTTATCCTGGAGATGGTGAAACTAACTACAATGGAAA
GTTCAAGGATAAAGCCACACTGACTGCAGACAAATCCTCCAGCACAGTCTACATGCAGCTCA
GCAGCCTAACATCTGAGGACTCTGCGGTCTACTTCTGTGCAAGAGCGGGTTATAGTGACTACG
TTTATGCTATGGACTACTGGGGTCAAGGAACCTCAGTCACCGTCTCCTCA
VL for mouse antibody D1D8H9F7
(SEQ ID NO.: 44)
EIVLTQSPALMAASPGEKVTIICSVSSSITSSNLHWYQQKSETSPKPWIYGTSNLASGVPVRFSGSGS
GTSYSLTISSMEAEDAATYYCQQWSSFPLTFGSGTKLEIK
(SEQ ID NO.: 46)
GAAATTGTGCTCACCCAGTCTCCAGCACTCATGGCTGCATCTCCAGGGGAGAAGGTCACCATC
ATCTGCAGTGTCAGCTCAAGTATAACTTCCAGCAACCTGCACTGGTACCAGCAGAAGTCAGA
AACCTCCCCCAAACCCTGGATTTATGGCACATCCAACCTGGCTTCTGGAGTCCCTGTTCGCTT
CAGTGGCAGTGGATCTGGGACCTCTTATTCTCTCACAATCAGCAGCATGGAGGCTGAAGATG
CTGCCACTTATTACTGTCAACAGTGGAGTACTTTCCCACTCACGTTCGGCTCGGGGACAAAGT
TGGAAATAAAA
VH-CDR1 for mouse antibody D1C2H1H1
(SEQ ID NO.: 47)
TYGMGIG
VH-CDR2 for mouse antibody D1C2H1H1
(SEQ ID NO.: 48)
SIWWDDNRYFNAALKS
VH-CDR3 for mouse antibody D1C2H1H1
(SEQ ID NO.: 49)
IEDGYYSYHSY
VL-CDR1 for mouse antibody D1C2H1H1
(SEQ ID NO.: 50)
KASQSVDFDGDSYMN
VL-CDR2 for mouse antibody D1C2H1H1
(SEQ ID NO.: 51)
AASNLVS
VL-CDR3 for mouse antibody D1C2H1H1
(SEQ ID NO.: 52)
QQIDEDPYT
VH for mouse antibody D1C2H1H1
(SEQ ID NO.: 53)
QVTLKESGPGILQPSQTLSLTCSFSGFSLSTYGMGIGWIRLPSGKGLEWLASIWWDDNRYFNAALK
SRLTISKDASKNQVFLKIASVDTADTATYYCARIEDGYYSYHSYWGQGTLVTVSA
(SEQ ID NO.: 55)
CAGGTTACTCTGAAAGAGTCTGGCCCTGGGATATTGCAGCCCTCCCAGACCCTCAGTCTGACT
TGTTCTTTCTCTGGGTTTTCACTGAGCACTTATGGCATGGGTATAGGCTGGATTCGTCTGCCTT
CGGGGAAGGGTCTGGAGTGGCTGGCAAGCATTTGGTGGGATGATAATAGATACTTTAACGCT
GCCCTGAAGAGCCGGCTCACTATCTCCAAGGATGCCTCCAAAAACCAGGTATTCCTCAAGAT
CGCCAGTGTGGACACTGCAGATACTGCCACATATTATTGTGCTCGAATAGAGGACGGCTATTA
TAGTTATCACTCTTACTGGGGCCAAGGGACTCTGGTCACTGTCTCTGCA
VL for mouse antibody D1C2H1H1
(SEQ ID NO.: 54)
DIVLTQSPASLAVSLGQRATISCKASQSYDFDGDSYMNWFQQKPGQPPKLLIYAASNLVSGIPARF
SGSGSGTDFTLNIHPVEEEDAATYYCQQIDEDPYTFGGGTKLEIE
(SEQ ID NO.: 56)
GACATTGTGCTGACCCAATCTCCAGCTTCTTTGGCTGTGTCTCTAGGGCAGAGGGCCACCATC
TCCTGCAAGGCCAGCCAAAGTGTTGATTTTGATGGTGATAGTTATATGAACTGGTTCCAACAG
AAACCAGGACAGCCACCCAAACTCCTCATCTATGCTGCATCCAATCTAGTATCTGGGATCCCA
GCCAGGTTTAGTGGCAGTGGGTCTGGGACAGACTTCACCCTCAACATCCATCCTGTGGAGGA
GGAGGATGCTGCAACCTATTACTGTCAGCAAATTGATGAGGATCCGTACACGTTCGGAGGGG
GGACCAAGCTGGAAATAGAA

Having thus described in detail preferred embodiments of the present disclosure, it is to be understood that the disclosure defined by the above paragraphs is not to be limited to particular details set forth in the above description as many apparent variations thereof are possible without departing from the spirit or scope of the present disclosure.

1. A bispecific antibody comprising two antigen-binding portions, comprising:
(a) a first antibody or antigen-binding portion or fragment thereof that binds TSLP comprising a heavy chain variable region (VH) comprising a VH CDR1 region, a VH CDR2 region and a VH CDR3 region, wherein the VH CDR1 region, the VH CDR2 region and the V_{H} CDR3 region comprise amino acid sequences of SEQ ID NOs: 1, 2 and 3, respectively, and
a light chain variable region (VL) comprising a VL CDR1 region, a VL CDR2 region and a VL CDR3 region, wherein the VL CDR1 region, the VL CDR2 region and the VL CDR3 region comprise amino acid sequences of SEQ ID NOs: 4, 5 and 6, respectively, and
(b) a second antibody or antigen-binding portion or fragment thereof that binds to a disease-specific protein.

2. The bispecific antibody of claim 1, wherein the second antibody or antigen-binding portion or fragment thereof comprises an antigen-binding portion of an anti-IgE antibody, an anti-IL4 antibody, an anti-IL4R antibody, an anti-IL13 antibody, an anti-IL13R antibody, an anti-IL5 antibody, an anti-IL5R antibody, an anti-IL17 antibody or an anti-TSLPR antibody.

3. The bispecific antibody of claim 1, which binds to TSLP and another disease-specific protein selected from the group consisting of IgE, Il-4, IL-13, IL-13R, Il-5, IL-5R, IL-17 or TSLPR.

4. The bispecific antibody of claim 1, which is a mouse, chimeric, humanized, or human antibody.

5. The bispecific antibody according to claim 1, wherein the first antibody further comprises:
(c) a heavy chain constant region comprising an amino acid sequence of SEQ ID NOs: 12, 13 or 57, linked to the heavy chain variable region; and
(d) a light chain constant region having an amino acid sequence of SEQ ID NO: 14, linked to the light chain variable region.

6. The bispecific antibody according to claim 5, wherein a C-terminus of the heavy chain variable region is linked to an N-terminus of the heavy chain constant region, and a C-terminus of the light chain variable region is linked to an N-terminus of the light chain constant region.

7. The bispecific antibody according to claim 5, wherein the heavy chain constant region comprises an amino acid sequence of SEQ ID NO: 12.

8. The bispecific antibody according to claim 5, wherein the first antibody is an IgGl isotype.

9. The bispecific antibody according to claim 5, wherein the first antibody or antigen-binding portion or fragment thereof is an scFv.

10. The bispecific antibody according to claim 5, wherein the second antibody or antigen-binding portion or fragment thereof is an scFv.

11. A pharmaceutical composition comprising a therapeutically effective amount of the bispecific antibody, or antigen-binding portion thereof, of claim 1, and a pharmaceutically acceptable carrier.

12. The pharmaceutical composition of claim 11, further comprising an anti-asthma agent, anti-ulcerative colitis drug, anti-atopic dermatitis drug, anti-allergic drug or anti-psoriasis drug.

13. The pharmaceutical composition of claim 12, wherein the anti-asthma agent is an antibody selected from the group consisting of an anti-TSLPR/IL7R antibody, an anti-IL4 antibody, an anti-IL4R antibody, an anti-IL-5 antibody, an anti-IL5R antibody, an anti-IL13 antibody, an anti-IL13R antibody, am anti-IL-17 antibody and an anti-IgE antibody.

14. A method for treating a disease associated with TSLP overexpression in a subject in need thereof, comprising administering to the subject the pharmaceutical composition of claim 11.

15. The method of claim 14, wherein the disease is asthma.

16. A vector containing a polynucleotide encoding the bispecific antibody, or the antigen-binding portion thereof, according to claim 1.

17. An isolated host cell containing the vector according to claim 16.

18. An isolated monoclonal antibody, or an antigen-binding portion thereof, which binds to thymic stromal lymphopoietin (TSLP), comprising
(a) a heavy chain variable region (VH) comprising a VH CDR1 region, a VH CDR2 region and a VH CDR3 region, wherein the VH CDR1 region, the VH CDR2 region and the VH CDR3 region comprise amino acid sequences of SEQ ID NOs: 1, 2 and 3, respectively, and
a light chain variable region (VL) comprising a VL CDR1 region, a VL CDR2 region and a VL CDR3 region, wherein the VL CDR1 region, the VL CDR2 region and the VL CDR3 region comprise amino acid sequences of SEQ ID NOs: 4, 5 and 6, respectively; or
(b) the VH region comprising an amino acid sequence of SEQ ID NO. 9, wherein X1=R, X2=V, and X3=R, and
the VL region comprising an amino acid sequence of SEQ ID NO. 11, wherein X1=A and X2=I.

19. The isolated monoclonal antibody, or the antigen-binding portion thereof, of claim 18, which (i) binds human TSLP; (ii) binds to monkey TSLP; and/or (iii) blocks human TSLP-human TSLPR/IL7R interaction.

20. The isolated monoclonal antibody, or the antigen-binding portion thereof, of claim 18, which is a mouse, chimeric, or humanized antibody.

21. The isolated monoclonal antibody, or the antigen-binding portion thereof, of claim 18, which is an IgGl, IgG2 or IgG4 isotype.
